(19) United States
(12) Patent Application Publication
(10) Pub. No.: US 2023/0272420 A1
(43) Pub. Date: Aug. 31, 2023

(54) RECOMBINANT VIRUSES, SURFACE-ENGINEERED DELIVERY SYSTEMS AND RELATED METHODS

(71) Applicant: DEVACELL, INC., San Diego, CA (US)

(72) Inventor: Inanc Ortac, San Diego, CA (US)

(21) Appl. No.: 18/007,902

(22) PCT Filed: Jun. 4, 2021
(86) PCT No.: PCT/US2021/036021

§ 371 Date: Dec. 2, 2022

Related U.S. Application Data
(60) Provisional application No. 63/193,014, filed on May 25, 2021.
(60) Provisional application No. 63/041,744, filed on Jun. 19, 2020.
(60) Provisional application No. 63/036,874, filed on Jun. 9, 2020.
(60) Provisional application No. 63/036,329, filed on Jun. 8, 2020.
(60) Provisional application No. 63/036,337, filed on Jun. 8, 2020.
(60) Provisional application No. 63/034,862, filed on Jun. 4, 2020.

(51) Int. Cl. C12N 15/86

(52) U.S. Cl. CPC C12N 15/86; C12N 2770/20022; C12N 2770/20034; C12N 2770/20043; C12N 2770/20051; C12N 2770/20061

(57) ABSTRACT

Provided herein are recombinant viruses and artificially coated delivery systems, and methods of use.

### TECHNICAL FIELD

The invention relates to novel recombinant viruses, artificially coated delivery systems, and related methods.

### BACKGROUND

There is a need for novel systemic delivery systems for pharmaceutical payloads, including recombinant viruses, nucleic acid vaccines, gene therapies and oncolytic virotherapies.

### SUMMARY

Provided herein is a surface-engineered delivery system, said system comprising:
a payload; and an artificial coating layer surrounding the payload, wherein the artificial coating is selected from the group consisting of: silica, titanium oxide and calcium phosphate.

In an embodiment, the payload is a recombinant virus having a recombinant genome or the recombinant genome itself, and the artificial coating layer surrounds the recombinant virus and/or recombinant genome.

In particular embodiments, the surface-engineered recombinant virus is at least one selected from the group consisting of: oncolytic viruses, enadenotucirev oncolytic virus, Poxviruses (including but not limited to VV), vaccinia virus, Herpes Virus, herpes simplex virus-1, herpes simplex virus-2, Rhabdoviruses (including but not limited to Rabies, VSV), Vesicular stomatitis virus, Coronavirus, SARS-CoV-2, Hepadnaviruses, Asfarviridae, Flaviviruses (including but not limited to Kunjin, West Nile, Dengue), Alphaviruses (including but not limited to SFV, SIN, VEE, M1), Togavirus, Hepatitis D virus, Orthomyxoviruses, Paramyxoviruses, Bunyaviruses, Filoviruses, Retroviruses (including but not limited to MMSV, MSCV), Noroviruses, adenoviruses (including but not limited to Ad5), adeno-associated viruses (including but not limited to AAV1, 2, 3, 4, 5, 6, 7, 8, 9), lentiviruses (including but not limited to HIV-1, HIV-2), measles virus, Newcastle disease virus, rotaviruses, poliovirus, Picornaviruses, enteroviruses, rhinoviruses, Coxsackie viruses, echoviruses, and hepatitis A virus. In other embodiments, the surface-engineered recombinant virus is an oncolytic virus and is selected from the group consisting of: NG-641 (PsiOxus), and Imlygic (talimogene laherparepvec). In a yet further embodiments, the recombinant virus and/or recombinant viral genome is used in a vaccine selected from AZD1222 (AstraZeneca), ChAdOx1-nCov19 (Oxford), Ad5-nCoV (CanSino), VSV, and Ad26 (J&J).

In another embodiment, the surface-engineered recombinant virus is a vaccine and the virus is a replication deficient Ad5 (Human) Adenovirus vector, wherein the recombinant genome encodes SARS-CoV-2 spike protein and the E1 & E3 genes are deleted. In yet another embodiment, the surface-engineered recombinant virus is oncolytic and the virus is VSV.

In another embodiment, the payload is at least one nucleic acid selected from mRNA, siRNA, miRNA, small nuclear RNA, plasmid DNA, antisense oligodeoxynucleotides, or any other nucleic acid known to those skilled in the art. In a further embodiment, the nucleic acid is complexed with a cation to form a polyion complex. In yet further embodiments the nucleic acid is selected from that contained in patisiran (Alnylam), BNT162b2 (Pfizer-BioNTech), and mRNA-1273 (Moderna).

In certain embodiments, the artificial coating is applied by conducting a charge-mediated sol-gel condensation reaction directly onto the surface of the payload. In further embodiments, the artificial coating comprises a silica gel matrix or titanium oxide gel matrix encapsulating the payload. In particular embodiments of the invention surface-engineered delivery system, the artificial coating comprises a cell-surface ligand selected from the group consisting of: proteins, polysaccharides, aptamers, peptides, oligonucleotides and small molecules. In other embodiments, the artificial coating comprises at least one targeting ligand selected from the group consisting of: Antibodies, transferrin, Hyaluronic acid, RGD (e.g., cRGD), IL4RPep-1, AS-1411, GBI-10, Folate, anisamide, and phenylboronic acid. In other embodiments, of the invention surface-engineered delivery system, the recombinant virus can be replication-competent or replication-defective. In certain embodiments, the recombinant virus has had its native envelope removed prior to coating with the artificial coating layer. In certain embodiments, the recombinant virus is natively non-enveloped.

Also provided herein is a surface-engineered recombinant virus vaccine, said virus comprising:
a recombinant Ad5 virus having a recombinant genome encoding SARS-CoV-2 spike protein, wherein at least a functional portion of E1 and E3 genes are deleted or wherein the genome does not contain any native Ad5 viral genes (i.e., “gutted” Ad5), and an artificial coating layer encapsulating the recombinant Ad5 virus, wherein said coating layer comprises an effective amount of a ligand to bind to a cell, and wherein the artificial coating is selected from the group consisting of: silica, titanium oxide and calcium phosphate.

Also provided herein is a method of making a surface-engineered recombinant virus, said method comprising:
producing a recombinant virus having a recombinant genome; and
applying an artificial coating to the recombinant virus, wherein the artificial coating is selected from the group consisting of: silica, titanium oxide and calcium phosphate. In particular embodiments, the artificial coating is applied by conducting a charge-mediated sol-gel condensation reaction directly onto the surface of the recombinant virus. In certain embodiments, the artificial coating comprises a silica gel matrix or titanium oxide gel matrix encapsulating the recombinant virus. In another embodiment, the recombinant virus has had it native-envelope removed prior to coating with the artificial coating layer. In another embodiment, the recombinant virus is natively non-enveloped.

Also provided herein, is a method of re-engineering the surface of a virus having a native-envelope, said method comprising:
removing the native-envelope from the virus to isolate a previously-enveloped-capsid; and applying an artificial coating to the previously-enveloped-capsid. Any enveloped virus known in the art is suitable for use herein. In certain embodiments, the enveloped virus can be selected from the group consisting of: Poxvirus, vaccinia virus, Herpes Virus, herpes simplex virus-1, herpes simplex virus-2, Rhabdovirus, Vesicular stomatitis virus, Coronavirus, SARS-CoV-2, Hepadnaviruses, Asfarviridae, Flavivirus, Alphavirus, Togavirus, Hepatitis D virus, Orthomyxovirus, Paramyxovirus, Bunyavirus, Filovirus, and Retrovirus. In particular embodiments, the native-envelope is removed or delipidated using a detergent and/or an extraction solvent. In some embodiments, the detergent and/or an extraction solvent can be selected from the group consisting of: Glutaraldehyde, chloroform, B-propiolactone, TWEEN-80, and dialkyl or trialkyl phosphates, alcohols, hydrocarbons, amines, ethers, n-butanol, di-isopropyl ether (DIPE), diethyl ether, either alone or in combination. In certain embodiments, the artificial coating is selected from the group consisting of: silica, titanium oxide and calcium phosphate. In one embodiment, applying the artificial coating further comprises conducting a charge-mediated sol-gel condensation reaction directly onto the surface of the previously-enveloped-capsid. In another embodiment, the artificial coating comprises a silica gel matrix or titanium oxide gel matrix encapsulating the previously-enveloped-capsid. In particular embodiments, the previously-enveloped-capsid is replication-competent or replication-defective.

Also provided herein is a surface-re-engineered virus comprising:
a previously-enveloped-capsid from a naturally occurring enveloped-virus; and an artificial coating layer surrounding the previously-enveloped-capsid. Any enveloped virus known in the art is suitable for use herein. In particular embodiments, the envelope virus can be selected from the group consisting of: Poxvirus, vaccinia virus, Herpes Virus, herpes simplex virus-1, herpes simplex virus-2, Rhabdovirus, Vesicular stomatitis virus (VSV), Coronavirus, SARS-CoV-2, Hepadnaviruses, Asfarviridae, Flavivirus, Alphavirus, Togavirus, Hepatitis D virus, Orthomyxovirus, Paramyxovirus, Bunyavirus, Filovirus, and Retrovirus. As set forth herein, the artificial coating is selected from the group consisting of: silica, titanium oxide and calcium phosphate. In certain embodiments, the artificial coating is applied by conducting a charge-mediated sol-gel condensation reaction directly onto the surface of the previously-enveloped-capsid. In other embodiments, the artificial coating comprises a silica gel matrix or titanium oxide gel matrix encapsulating the previously-enveloped-capsid. The previously-enveloped-capsid can be either replication-competent or replication-defective.

Also provided herein is a method of re-engineering a virus having a native-envelope, said method comprising: removing the native-envelope surrounding a capsid from the virus; isolating the previously-enveloped-capsid; and applying an artificial coating to the previously-enveloped-capsid.

Also provided herein is a composition comprising:
a capsid from a native envelope-virus, wherein the capsid is devoid of its native envelope; and an artificial coating-layer, wherein the coating-layer encapsulates the capsid. The native envelope virus for use herein can be any enveloped virus known in the art or described herein. In certain embodiments, the envelope-virus is selected from the group consisting of: Herpesviruses, Poxviruses (e.g., vaccinia virus), Hepadnaviruses, Asfarviridae, Flavivirus, Alphavirus, Togavirus, Coronavirus, Hepatitis D, Orthomyxovirus, Paramyxovirus, Rhabdovirus, Bunyavirus, Filovirus, and Retroviruses.

Also provided herein is a method of making a surface-engineered natively non-enveloped virus, said method comprising: providing a natively non-enveloped virus and applying an artificial coating to the natively non-enveloped virus.

Also provided herein is a composition comprising:
a natively non-enveloped virus; and an artificial coating-layer, wherein the coating-layer encapsulates the natively non-enveloped virus. The natively non-enveloped virus for use herein can be any natively non-enveloped virus known in the art or described herein. In certain embodiments, the natively non-enveloped virus is at least one selected from the group consisting of: adeno-associated virus (including but not limited to AAV2, 3, 5, 6, 8, 9) and adenovirus (including but not limited to Ad5).

Also provided herein is a method of providing a surface coating over a nucleic acid, said method comprising: complexing the nucleic acid with a cation to provide a cationic polyion complex, and applying an artificial coating to the cationic polyion complex.

Also provided herein is a composition comprising:
a nucleic acid contained in a cationic polyion complex; and an artificial coating-layer, wherein the coating-layer encapsulates the nucleic acid contained in the polyion complex. In certain embodiments, the nucleic acid is mRNA, siRNA, miRNA, small nuclear RNA, plasmid DNA, antisense oligodeoxynucleotides, or any other nucleic acid known to those skilled in the art. In certain embodiments, the cation is a cationic polymer such as polyethylenimine or any cationic polymer known to those of skill in the art to be able to generate cationic polyion complexes with nucleic acids. In certain embodiments, the cation is a cationic lipid, such as any cationic lipid known to those of skill in the art to be able to generate cationic complexes with nucleic acids. In certain embodiments, the mRNA encodes patient-specific neoantigens to enable personalized anti-cancer therapy.

In each of the invention coated delivery systems provided herein, the coating-layer protects the viral capsid, non-enveloped virion, or nucleic acid from immune recognition and neutralization during therapy; and/or improve pharmacokinetics in blood/circulation; and/or improves accumulation in target tissue; and/or prevents opsonization and rapid clearance upon systemic administration. In certain embodiments of the invention coated delivery systems provided herein, the coating-layer further comprises binding agents (e.g., targeting-ligands) on its surface that changes the uptake and/or biological activity and/or cell/tissue targeting of the payload in the surface-engineered delivery system relative to the non-coated payload, particularly of the surface-engineered recombinant virus relative to the native envelope-virus.

Also provided herein is a method of making a surface-re-engineered recombinant virus, comprising removing the envelope of an envelope virus to produce a envelope-free-capsid; and encapsulating the envelope-free-capsid with an artificial coating-layer.

### BRIEF DESCRIPTION OF THE FIGURES

FIG. 1 shows the process for generating the inventive surface-engineered recombinant virus, where a coating is generated over a recombinant virus by first applying an organic coating and then inducing a sol-gel reaction. Once the surface of the virus has been engineered by coating with the sol-gel, ligands and other functional molecules are attached to the surface using PEG spacers to provide new functionality. The figure shows coating of adenovirus, but this method is applicable to all viruses. For enveloped viruses, a step can be applied to strip the virus of its envelop before applying the coating.

FIG. 2 shows electron microscope images of surface-engineered vaccinia virus and adenovirus generated using the inventive coating. The coating utilizes inorganic/organic hybrid chemistry, with one-step synthesis at room temperature and neutral pH with a ˜4 hour reaction duration. This enables very flexible surface functionalization chemistry that allows high-density attachment of small molecules, peptides, oligos, proteins, sugars or other small/large molecules. The coating provides 10-100 fold improvement of cancer cell infectivity and 10-100 fold improvement of blood circulation half life. The coating is characterized by optimized and scalable chemistry with great batch-to-batch consistency, and can be applied to both enveloped and non-enveloped viruses. The coating has thus far been applied to Vaccinia Virus, Adenovirus, Measles Virus, Herpes Simplex Virus, Retroviral Replicating Virus, and Vesicular Stomatitis Virus, and is thus is easily applicable to any virus.

FIG. 3 shows a schematic representation of the benefits provided by the surface coating on the surface-engineered recombinant virus in vivo. The coating prevents surface access of antibodies to prevent neutralization of the virus, thus allowing systemic administration and repeated administration. The coating reduces liver uptake and prevents clearance by the reticuloendothelial system, thus prolonging blood circulation. The coating allows attachment of targeting molecules to achieve tissue targeting.

FIG. 4 shows the scheme for large-scale GMP-grade manufacturing of the surface-engineered recombinant virus. The coating reaction is a bulk a bulk reaction performed at 25° C., and the total reaction takes 1 hour. The reaction is performed at neutral pH in aqueous conditions. It is a simple one-step reaction easily implemented during the last stage of viral vector production. The coating is self-assembling, and the process is self-terminating without any other reagents required.

FIG. 5 shows the uptake and delivery mechanism for the surface-engineered recombinant virus when folate is attached as an exemplary targeting molecule. The folate attached to the surface of the surface-engineered virus binds to the folate receptor (which is highly present on cancer cells), stimulating receptor-mediated endocytosis of the surface-engineered recombinant virus. The coating degrades in the endosome, resulting in acidification of the endosome that triggers endosomolysis, releasing the virus into the cytosol. The now-uncoated naked virus is fully functional and now behaves the same as the native virus upon viral entry into the cell; it inserts its genome into the cell nucleus and replicates. Eventually, the viral replication causes the cell to lyse and release the newly produced viral particles. Other receptors and other uptake mechanisms can be utilized depending on the nature of the ligand attached to the surface-engineered recombinant virus.

FIG. 6 shows a comparison of uptake mechanisms between the surface-engineered recombinant virus and native enveloped and non-enveloped viruses. When a ligand molecule is attached to its surface, the surface-engineered recombinant virus utilizes receptor-mediated endocytosis similarly to a non-enveloped virus. By changing the ligand, the cell/tissue tropism of the surface-engineered recombinant virus can be changed. Functional molecules can also be attached to the surface-engineered recombinant virus to enhance endosomal rupture/escape or nuclear transport and entry.

FIG. 7 shows that the inventive coating does not negatively affect the infectivity of the virus; reporter protein expression at 4 days after infection in vitro was similar between naked and coated vesicular stomatitis virus.

FIG. 8 shows that the inventive coating increases viral infectivity, with reporter protein activity after in vitro Ad5 infection increased by nearly two fold.

FIG. 9 shows that the inventive coating protects the virus from antibody neutralization, with reporter protein expression maintained after Ad5 virus infection even in the presence of antibody concentrations that completely neutralized non-coated Ad5 virus.

FIGS. 10A and 10B show the immune stealth property conferred by the inventive coating. The coating reduced the in vitro macrophage response to Ad5 virus, even out to 80 days, as measured by macrophage GM-CSF and IL-10 secretion. This indicates that the coating reduced the recognition of the virus by macrophages.

FIGS. 11A and 11B show that attaching a ligand to the surface-engineered recombinant virus allows cell-specific targeting. Specifically, tethering of folate to the surface of the surface-engineered recombinant Ad5 virus resulted in increased reporter gene expression in U2OS cells with high folate receptor expression and reduced reporter gene expression in H226 cells with low folate receptor expression when compared to non-coated Ad5 virus. The increased expression was due to the presence of folate on the surface of the surface-engineered virus, whereas the reduced expression was due to the coating and PEG layer preventing nonspecific cell entry.

FIGS. 12A and 12B show that the coating increases the in vivo circulation time for the surface-engineered recombinant virus. The coating increased the circulation time for adenovirus compared to non-coated adenovirus after tail vein injection in mice, in both naïve mice and mice that had been previously immunized with a live virus. The coating thus prevented the adenovirus from being cleared from the circulation, even after previous immunization.

FIG. 12C shows that the coating allows repeated administration of the surface-engineered recombinant virus. Repeated immunization of mice with a coated Ad5 vaccine, with three intramuscular administrations one week apart, resulted in further increases in serum neutralizing antibody titer with each successive injection.

FIGS. 13A and 13C show that the coating allows anti-tumor treatment at lower doses of the surface-engineered recombinant virus in vivo. In tumor-implanted immunodeficient mice receiving an anti-tumor vaccinia virus, coating of the virus still reduced tumor size at only 30% of the conventional dose, and the tumor size reduction at the lower dose was greater than that observed with the non-coated virus.

FIGS. 13B and 13D show that the coating allows anti-tumor treatment at lower doses of the surface-engineered recombinant virus in vivo. In tumor-implanted immunodeficient mice receiving an anti-tumor vaccinia virus, coating of the virus still increased survival at only 30% of the conventional dose, and the survival benefit at the lower dose was greater than that observed with the non-coated virus.

FIG. 14A shows that coating of a vaccine results in stronger humoral immunity. Coating of Ad5 virus expressing the COVID-19 spike protein resulted in increased IgG antibody generation in mice, even out to day 79, after three intramuscular injections, two weeks apart.

FIG. 14B shows that coating of a vaccine results in stronger humoral immunity. Coating of Ad5 virus expressing the COVID-19 spike protein resulted in increased CD8+ lymphocyte levels in mice after 3 intramuscular injections, two weeks apart.

FIG. 15 shows that coating of a protein antigen as the payload results in prolonged tissue residence, even out to day 45.

FIG. 16 shows that coating of a virus produces a stronger anti-tumor effect in vivo compared to non-coated virus when the virus is injected at a suboptimal dose. Immunocompetent tumor-implanted mice injected with coated Ad5 virus showed reduced tumor growth compared to mice injected with non-coated Ad5 virus.

### DETAILED DESCRIPTION

Provided herein is a surface-engineered delivery system, said system comprising:
a payload; and an artificial coating layer surrounding the payload, wherein the artificial coating is selected from the group consisting of: silica, titanium oxide and calcium phosphate.

In an embodiment, the payload is a recombinant virus having a recombinant genome or the recombinant genome itself, and the artificial coating layer surrounds the recombinant virus and/or recombinant genome.

In another embodiment, the payload is at least one nucleic acid selected from mRNA, siRNA, miRNA, small nuclear RNA, plasmid DNA, antisense oligodeoxynucleotides, or any other nucleic acid known to those skilled in the art. In a further embodiment, the nucleic acid is complexed with a cation to form a polyion complex. In certain embodiments, the cation is a cationic polymer such as polyethylenimine or any cationic polymer known to those of skill in the art to be able to generate cationic polyion complexes with nucleic acids. In certain embodiments, the cation is a cationic lipid, such as any cationic lipid known to those of skill in the art to be able to generate cationic complexes with nucleic acids.

As used herein, the phrase “surface-engineered delivery system” refers to any delivery system where an artificial coating layer encapsulates a naturally occurring or recombinant virus, or a nucleic acid. The invention surface-engineered delivery systems provided herein can function as one or more of a vaccine, immunotherapy, oncolytic virus, nucleic acid medicine, or viral or nonviral gene therapy.

As used herein, the phrase “surface-engineered recombinant virus” refers to any recombinant virus that contains an artificial coating layer encapsulating either the entire native-virus or naturally occurring virus, whether enveloped or non-enveloped, or encapsulating a previously-enveloped-capsid isolated from a native-virus. The invention surface-engineered recombinant viruses provided herein can function as one or more of a vaccine, oncolytic virus and/or a viral vector or vehicle for gene therapy.

As used herein, a “native-virus” or “naturally occurring virus” refers to any virus, wild-type or recombinant, that is produced in a cell and either buds from a cell or can be isolated once a cell lyses.

As used herein, “natively non-enveloped virus” refers to any virus, wild-type or recombinant, that does not naturally contain an envelope and is produced in a cell and either buds from a cell or can be isolated once a cell lyses.

As used herein, the phrase “recombinant virus” or “viral vector” refers to any virus, whether enveloped or non-enveloped, that has had its genome manipulated or engineered, such as by the insertion or deletion of particular nucleic acid regions resulting in a “recombinant genome.” For example, deletion of viral genes that are essential for viral replication results in replication-defective recombinant viruses. Recombinant viruses can also contain nucleic acid insertions coding for functional therapeutic proteins to replace the corresponding defective proteins, or coding for selectable markers, or the like.

Suitable recombinant viruses (viral vectors) for use herein for surface-engineering, include all recombinant viruses, whether replication-competent, replication-defective, enveloped or non-enveloped, such as those that are currently in clinical trials or commercially available, such as: Enadenotucirev oncolytic virus (i.e., NG-641; PsiOxux; see US 20190233536 A1, which is incorporated herein by reference in its entirety for all purposes), Ad26 (Janssen/J&J), Imlygic (Amgen), and the like. In particular embodiments, the surface-engineered recombinant virus is selected from the group consisting of: enadenotucirev oncolytic virus, Poxvirus, vaccinia virus, Herpes Virus, herpes simplex virus-1, herpes simplex virus-2, Rhabdovirus, Vesicular stomatitis virus, Coronavirus, SARS-CoV-2, Hepadnaviruses, Asfarviridae, Flavivirus, Alphavirus, Togavirus, Hepatitis D virus, Orthomyxovirus, Paramyxovirus, Bunyavirus, Filovirus, Retrovirus, Noroviruses, adenoviruses, rotaviruses, poliovirus, Picornaviruses, enteroviruses, rhinoviruses, Coxsackie viruses, echoviruses, hepatitis A virus, adeno-associated virus, lentiviruses, measles virus, and Newcastle disease virus.

In other embodiments, the surface-engineered recombinant virus is an oncolytic virus and is selected from the group consisting of: NG-641 (PsiOxus), Imlygic (talimogene laherparepvec), Pexa-Vec (Transgene/Sillajen), Reolysin (Oncolytics Biotech), DS-1647 (Daiichi Sankyo), TG1042 (Transgene/Ascend), Cavatak (Merck), GL-ONC1 (Genelux), Marabex (Turnstone/Abbvie), ORCA-010 (Orca/VCN), ParvOryx (Oryx), LOAd703 (Lokon Pharma), PV701 (Wellstat Group), MV-NIS (Vyriad), ONCOS-102 (Targovax), Seprehvir (Sorrento), Enadenotucirev (Psioxus), CG0070 (Cold Genesys), Telomelysin (Oncolys Biopharma), JX-929 (Sillajen), VSV Cancer Project (AstraZeneca), Ad-VirRx 007 (Multivir), NG-348 (Psioxus/BMS), VSV-GP (Viratherapeutics), RP1/RP2/RP3 (Replimune) and WO-12 (Western Oncolytics/Pfizer).

In a yet further embodiment, the surface-engineered recombinant virus is a vaccine and is selected from AZD1222 (AstraZeneca), ChAdOx1-nCov19 (Oxford), Ad5-nCoV (CanSino), VSV, and Ad26 (J&J).

In another embodiment, the surface-engineered recombinant virus and/or recombinant viral genome is an adeno-associated virus of any known serotype, including but not limited to AAV1, 2, 3, 4, 5, 6, 7, 8, and 9. Because adeno-associated virus is a non-enveloped virus, the surface engineering of the present invention includes applying the inventive coating to the surface of the capsid of an adeno-associated virus using the methods described herein. Native AAV1, 2, 4, 5, 8, and 9 are known to infect central nervous system tissue. Native AAV1, 8, and 9 are known to infect heart tissue. Native AAV2 is known to infect kidney tissue. Native AAV7, 8, and 9 are known to infect liver tissue. Native AAV4, 5, 6, and 9 are known to infect lung tissue. Native AAV8 is known to infect pancreatic tissue. Native AAV2, 5, and 8 are known to infect photoreceptor cells. Native AAV1, 2, 4, 5, and 8 are known to infect retinal pigment epithelial tissue. Native AAV1, 6, 7, 8, and 9 are known to infect skeletal muscle tissue. In another embodiment, the surface-engineered recombinant virus is a pseudotyped adeno-associated virus, where the viral capsid and genome are from different viral serotypes. For example, AAV2/5 has the genome of serotype 2 packaged in the capsid from serotype 5. AAV2/5 targets neurons that are not efficiently transduced by AAV2/2, and is distributed more widely in the brain, indicating improved transduction efficiency. In another embodiment, the surface-engineered recombinant virus is an adeno-associated virus with a hybrid capsid derived from multiple different serotypes. One common example is AAV-DJ, which contains a hybrid capsid derived from eight serotypes. AAV-DJ displays a higher transduction efficiency in vitro than any wild type serotype; in vivo, it displays very high infectivity across a broad range of cell types. The mutant AAV-DJ8 displays the properties of AAV-DJ, but with enhanced brain uptake. The surface-engineered recombinant adeno-associated virus and/or viral genome of the present invention can be used deliver genes to any of the above tissues or cells, including in vivo. The surface-engineered recombinant adeno-associated virus and/or viral genome can be used to deliver genes to any tissue type known to those of skill the art to be targeted by native adeno-associated virus. Through engineering of the surface to contain appropriate ligands known to those of skill in the art, the surface-engineered recombinant adeno-associated virus and/or viral genome can be used to deliver genes to any cell or tissue type not normally targeted by the native adeno-associated virus. In another embodiment, the surface-engineered recombinant virus or viral genome is self-complementary AAV (scAAV). scAAV contains complementary sequences that are capable of spontaneously annealing, upon infection, which eliminates the requirement for host cell DNA synthesis. In another embodiment, the surface-engineered recombinant virus or viral genome has increased packaging capacity, for example through concatemer formation or homologous recombination as known to those of skill in the art.

The surface engineering of the recombinant adeno-associated virus and/or viral genome in any of the embodiments above can enhance in vivo transduction and/or enable repeated in vivo administration of the recombinant adeno-associated virus, including with systemic administration, because the coating of the virus protects the virus against clearance by the immune system and/or protects the virus from binding of neutralizing antibodies. Thus, the surface-engineered recombinant adeno-associated virus or viral genome can be used, including for systemic delivery, even when the recipient has pre-existing immunity and/or pre-existing antibodies against adeno-associated virus. The surface-engineered recombinant adeno-associated virus or viral genome can be used for multiple administrations to increase the amount and/or duration of transgene expression, even when the first administration of a native, non-surface-engineered recombinant adeno-associated virus would normally induce an immune response that limits the effectiveness of subsequent administrations. The surface-engineered recombinant adeno-associated virus and/or viral genome in any of the embodiments above can be used as a gene therapy or as a vaccine. The surface-engineered recombinant adeno-associated virus and/or viral genome in any of the embodiments above can be used as a vector to deliver genes encoding components for genome editing, such as Cas, TALEN, or zinc finger nucleases.

In yet further embodiments, the recombinant virus and/or recombinant viral genome is a gene therapy selected from Onasemnogene Abeparvovec-Xioi (AveXis/Novartis), Voretigene neparvovec-rzyl (Spark), MB-107 (Mustang Bio), AMT-061 (uniQure), PTC-AADC (PTC Therapeutics), ALD-104 or Starbeam ALD-102 (Bluebird Bio), VB-111 (Vascular Biogenics), EB-101 (Abeona Therapeutics), BIIB-111 (Biogen), BENEGENE-2 (Spark/Pfizer), BIIB112 (Biogen), SRP-9001 (Sarepta), BMN-270 (BioMarin), OXB-102 (Oxford BioSciences), HMI-102 (Homology Medicines), RP-A501 (Rocket Pharmaceuticals), LB-001 (LogicBio Therapeutics), Ad-RTS-hIL-12 (ZIOPHARM Oncology), SGT-001 (Solid Biosciences), B-VEC (Krystal Biotech), SRP-9003 (Sarepta), RG6357 (Roche/Spark), MYO-201 (Sarepta), RGX-314 (RegenXBio), AAV-GAD (MeiraGTx), MYO-102 (Sarepta), DTX401 (Ultragenyx), VY-AADC (Neurocrine/Voyager), AAV-AQP1 (MeiraGTx), EDIT-101 (Editas/Allergan), DTX301 (Ultragenyx), ADVM-022 (Adverum), RGX-111, RegenXBio, OXB-201 (Oxford BioMedica), AT132 (Axovant/Astellas), AVXS-201 (AveXis/Novartis), ABO-102 (Abeona), ST-920 (Sangamo), AT-GTX-501 (Amicus), AT-GTX-502 (Amicus), ACHM-CNGB3 (Applied Genetic Technologies), AGTC-402 (Applied Genetic Technologies), AXO-AAV-GM1 (Axovant), AGTC-501 (Applied Genetic Technologies), ABO-101 (Abeona), SB-318 (Sangamo), AXO-AAV-GM2 (Axovant), AAV-RPE65 (MeiraGTx), RG6367 (Roche/Spark), RGX-121 (RegenXBio), RGX-501 (RegenXBio), RG6358 (Roche/Spark), MYO-301 (Sarepta), HMI-103 (Homology Medicines), LB-101 (LogicBio), HMI-202 (Homology Medicines), AVR-RD-03 (Avrobio), MYO-103 (Sarepta), and BBP-631 (BridgeBio Pharma).

Exemplary enveloped viruses or lipid containing viruses contemplated for use as recombinant viruses herein include: Poxvirus (e.g., vaccinia virus, and the like), Herpes Virus (e.g., herpes simplex, and the like), Rhabdovirus (e.g., Vesicular stomatitis virus, and the like), Coronavirus (e.g., SARS-CoV-2, and the like), Hepadnaviruses, Asfarviridae, Flavivirus, Alphavirus, Togavirus, Hepatitis D, Orthomyxovirus, Paramyxovirus, Bunyavirus, Filovirus, Retroviruses, Alphavirus (alphaviruses), Rubivurus (rubella virus), Flavivirus (Flaviviruses), Pestivirus (mucosal disease viruses), hepatitis C virus, Coronavirus, (Coronaviruses), severe acute respiratory syndrome (SARS), Torovirus, (toroviruses), Arteivirus, (arteriviruses), Paramyxovirus, (Paramyxoviruses), Rubulavirus (rubulavriuses), Morbillivirus (morbillivuruses), Pneumovirinae (the pneumoviruses), Pneumovirus (pneumoviruses), Vesiculovirus (vesiculoviruses), Lyssavirus (lyssaviruses), Ephemerovirus (ephemeroviruses), Cytorhabdovirus (plant rhabdovirus group A), Nucleorhabdovirus (plant rhabdovirus group B), Filovirus (filoviruses), Influenzavirus A, B (influenza A and B viruses), Influenza virus C (influenza C virus), (unnamed, Thogoto-like viruses), Bunyavirus (bunyaviruses), Phlebovirus (phleboviruses), Nairovirus (nairoviruses), Hantavirus (hantaviruses), Tospovirus (tospoviruses), Arenavirus (arenaviruses), unnamed mammalian type B retroviruses, unnamed, mammalian and reptilian type C retroviruses, unnamed, type D retroviruses, Lentivirus (lentiviruses), Spumavirus (spumaviruses), Orthohepadnavirus (hepadnaviruses of mammals), Avihepadnavirus (hepadnaviruses of birds), Simplexvirus (simplexviruses), Varicellovirus (varicelloviruses), Betaherpesvirinae (the cytomegaloviruses), Cytomegalovirus (cytomegaloviruses), Muromegalovirus (murine cytomegaloviruses), Roseolovirus (human herpes virus 6, 7, 8), Gammaherpesvirinae (the lymphocyte-associated herpes viruses), Lymphocryptovirus (Epstein-Barr-like viruses), Rhadinovirus (saimiri-ateles-like herpes viruses), Orthopoxvirus (orthopoxviruses), Parapoxvirus (parapoxviruses), Avipoxvirus (fowlpox viruses), Capripoxvirus (sheeppox-like viruses), Leporipoxvirus (myxomaviruses), Suipoxvirus (swine-pox viruses), Molluscipoxvirus (molluscum contagiosum viruses), Yatapoxvirus (yabapox and tanapox viruses), Unnamed, African swine fever-like viruses, Iridovirus (small iridescent insect viruses), Ranavirus (front iridoviruses), Lymphocystivirus (lymphocystis viruses of fish), Togaviridae, Flaviviridae, Coronaviridae, Enabdoviridae, Filoviridae, Paramyxoviridae, Orthomyxoviridae, Bunyaviridae, Arenaviridae, Retroviridae, Hepadnaviridae, Herpesviridae, Poxviridae, and any other lipid-containing, enveloped virus. In certain embodiments, an enveloped recombinant virus has had its native-envelope removed prior to coating with the artificial coating layer.

Exemplary natively non-enveloped viruses contemplated for use as recombinant viruses herein include adeno-associated viruses, adenoviruses, noroviruses, rhinoviruses, polioviruses, coxsackieviruses, rotaviruses, hepatitis A virus, flock house virus, reoviruses, and papillomaviruses.

In another embodiment, the surface-engineered recombinant virus is a vaccine and the virus is a replication deficient Ad5 (Human) Adenovirus vector, wherein the recombinant genome encodes SARS-CoV-2 spike protein and the E1 & E3 genes are deleted, or the recombinant genome does not contain any native Ad5 viral genes. In yet another embodiment, the surface-engineered recombinant virus is oncolytic and the virus is VSV.

In yet further embodiments, the payload is a nucleic acid, and the nucleic acid is selected from that contained in QR-110 (ProQR therapeutics), Neovasculgen (Human Stem Cells Institute), ND-L02-s0201 (Nitto Denko/BMS), HGF plasmid (AnGes), TAVO (Oncosec), GEN-1 (Celsion), QR-313 (Wings Therapeutics), INXN-4001 (Triple-Gene), AAT genome editing (Intellia), QR-411 (ProQR Therapeutics), patisiran, givosiran, lumasiran, vultisiran, cemdisiran, inclisiran, ALN-AAT02, ALN-AGT, ALN-HSD (Alnylam), ALN-COV, ALN-HBV02 (Alnylam/Vir), BNT162b2 (Pfizer-BioNTech), mRNA-1273, mRNA-1647, mRNA-1653, mRNA-1893, mRNA-1345, mRNA-1189, mRNA-1010, mRNA-1020, mRNA-1030, mRNA-1644, mRNA-1574, mRNA-1215, mRNA-1851, mRNA-1944, mRNA-0184, mRNA-6981, mRNA-6231, mRNA-4157, mRNA-5671, mRNA-2416, mRNA-2752, MEDI1191, AZD8601, mRNA-3927, mRNA-3705, mRNA-3283, mRNA-3745 (Moderna), MRT5005, MRT5500 (TranslateBio). In certain embodiments, the payload is mRNA, and the mRNA encodes patient-specific neoantigens to enable personalized anti-cancer therapy.

As used herein, the phrase “artificial coating layer” refers to any biocompatible material that functions to encapsulate or surround the payload, i.e., the recombinant virus, viral capsid, or nucleic acid. In each of the invention coated delivery systems provided herein, the artificial coating-layer protects the recombinant virus or the previously-enveloped-capsid or the nucleic acid from immune recognition and neutralization during therapy. In particular embodiments, the artificial coating is selected from the group consisting of: silica, titanium oxide and calcium phosphate. In certain embodiments, the artificial coating is applied by conducting a charge-mediated sol-gel condensation reaction directly onto the surface of the recombinant virus. In further embodiments, the artificial coating comprises a silica gel matrix or titanium oxide gel matrix encapsulating the recombinant virus.

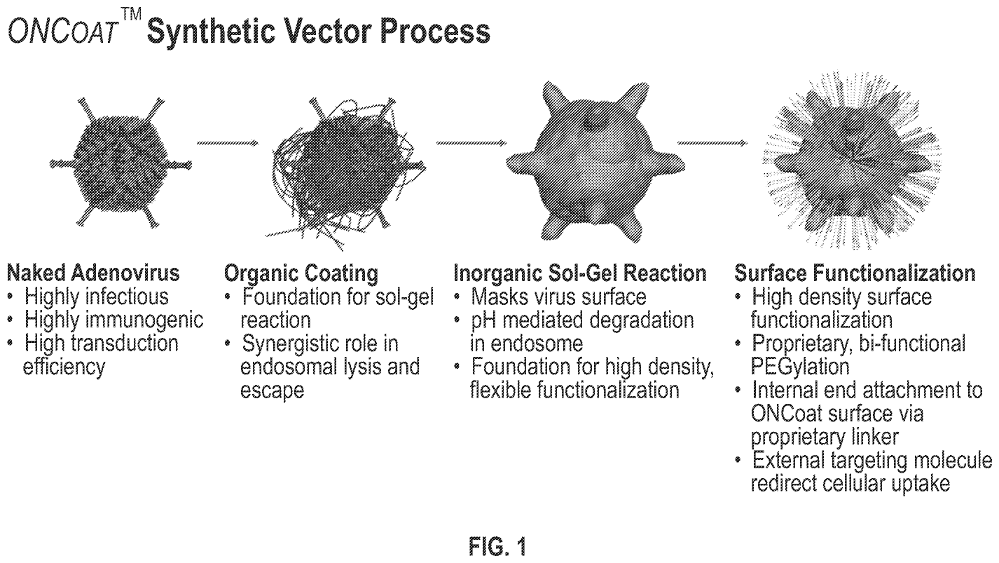

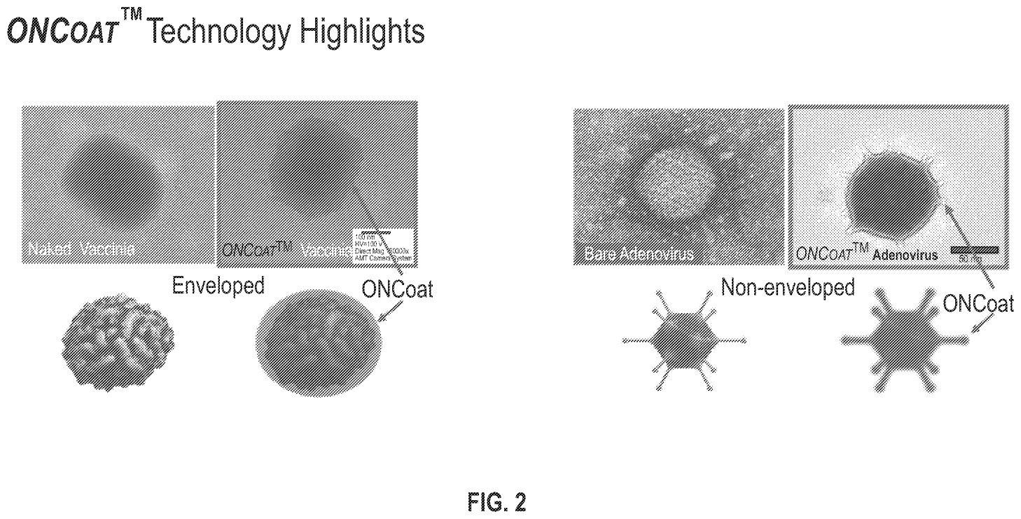

In particular embodiments for applying the artificial coating layer, as set forth in the methods described in US 2019/0151253A1, the virus or viral capsid is incubated with polycationic polymer to shift the virus surface charge towards the positive direction. Next, a silica precursor, such as tetramethyl (or tetraethyl) orthosilicate, and the like, is hydrolyzed to generate silicic acid, which is added into a suspension containing the virus or viral capsid surface-modified with polycationic polymer. The polymer on the virus surface templates the polycondensation reaction (sol-gel) to produce silica gel. In particular embodiments, the surface can then further functionalized with stabilization and targeting ligands. The scheme for applying the coating layer and stabilization and targeting ligands is shown in FIG. 1. FIG. 2 shows electron microscope images of surface-engineered vaccinia virus and adenovirus generated using the inventive coating.

In the case of a nucleic acid, the nucleic acid can be complexed with a cation such as a polycationic polymer or cationic lipid to form a positively charged polyion complex. Methods for such complexation are described, for example, by de Ilarduya (Eur J Pharm Sci. 2010 Jun. 14; 40(3):159-70) and are well known to those of skill in the art. The silica precursor is then added to a suspension containing the polyion complex containing the nucleic acid, and the surface of the polyion complex templates the polycondensation reaction (sol-gel) to produce silica gel. In particular embodiments, the surface can then further functionalized with stabilization, targeting, an uptake-enhancing ligands.

The polycationic polymer used for any embodiment herein can be any biomedically suitable polycationic polymer known to those of skill in the art to electrostatically interact with negatively charged molecules or surfaces, including but not limited to poly-L-lysine (PLL), polyarginine, polyethyleneimine (PEI), polyallylamine, polyamines, diethylaminoethyl-dextran (DEAE-dextran), branched polymers such as poly(amidoamine) (PAMAM) dendrimers, Tfx-50, dioctadecylamidoglycylspermine, or positively charged polypeptides. The cationic lipid can be any biomedically suitable cationic lipid polycationic polymer known to those of skill in the art to electrostatically interact with negatively charged molecules or surfaces such as MVLS, DOTMA, ethyl PC, DDAB, DOTAP, DC-cholesterol, GL67, or DODMA. Any suitable combination of cationic polymers can be used. Any suitable combination of cationic lipids can be used. Any suitable combination of cationic polymers and lipids can be used.

The artificial coating used for any embodiment herein is preferably silica, titanium oxide, or calcium phosphate. The artificial coating is particularly preferably formed from a silica gel, titanium oxide gel, or calcium phosphate gel formed through a sol-gel condensation reaction. The calcium phosphate can be hydroxyapatite, -tricalcium phosphate, biphasic calcium phosphate or any other suitable calcium phosphate. However, the artificial coating is not limited to these materials, and any other material known to those of skill in the art to form gels through sol-gel condensation reactions and to be suitable for biomedical use can be used as the artificial coating. Zinc oxide, magnesium oxide, calcium oxide, zirconium oxide, aluminum oxide, iron oxide, tungsten oxide, cerium oxide, tin oxide, or any other suitable metal oxide can also be the artificial coating material. Any combination of suitable materials can also be used as the coating material.

In some embodiments, for example, an exemplary silica-based or titanium oxide-based artificial coating-layer can be formed by the direct condensation of a removable silica matrix or titanium oxide matrix on the surface of the recombinant viruses or previously-enveloped-viral capsids to form the surface-engineered-recombinant virus while preserving biological activity. Using the disclosed fabrication techniques, for example, the exemplary silica matrix or titanium oxide matrix can be formed directly on the surface of the recombinant viruses or previously-enveloped-capsids under suitable reaction conditions. This allows higher encapsulation efficiency without consequent loss of activity of the recombinant virus or viral capsids. This also brings fine control over particle size giving surface-engineered-recombinant viruses with well-defined size characteristics. For example, silica or titanium oxide can be used to form the artificial coating-layer due to its biocompatibility and biodegradability.

In other embodiments, the disclosed technology can include a sensitizing agent within the synthesized surface-engineered delivery system to allow externally triggered release of the encapsulated payload. The sensitizing agent can be fluorocarbon emulsions as ultrasound cavitation centers. The sensitizing agent can be pH-responsive. Also provided herein are exemplary methods for the surface-functionalization of the artificial-coating-layer for, e.g., functionalizing of the silica or titanium surface to improve circulation time (increase biological half-life), tumor and/or antigen targeting, cell/tissue targeting, cell uptake and the like.

In another embodiment, the exemplary method for the encapsulation process of a recombinant virus or a previously-enveloped-capsid that preserves the gene transcription activity of the encapsulated recombinant virus or engineered-naked-capsids includes forming an intermediate biomaterial by binding a surface-charged material with a naked-viral-capsid from a non-enveloped virus or a previously-enveloped-capsid, such that the formed intermediate biomaterial comprises regions having a net surface charge, e.g., which may be different from the original surface charge on the capsid. For example, in a particular embodiment, a negatively charged naked-viral-capsid (e.g., from a non-enveloped virus or a previously-enveloped-capsid) is electrostatically reacted with a cationic polymer, poly-L-lysine (PLL), to modify the surface charge on the viral-capsid, e.g., the PLL is bound to the surface of the naked-viral-capsid by an electrostatic force. The invention method includes forming a surface-engineered-recombinant virus by forming an artificial coating-layer to encapsulate the intermediate biomaterial (e.g., the cationic polymer and PLL), in which the encapsulated viral-capsid maintains its ability to regulate gene expression (e.g., via gene transcription). For example, in one embodiment where silica is utilized, the exemplary positively charged viral-material structure attracts negatively charged silica precursor and hydroxyl ions creating a basic environment suitable for a silica polycondensation reaction to form the viral-capsid coating-layer (e.g., silica-based nanoparticle) that encapsulates the viral capsid.

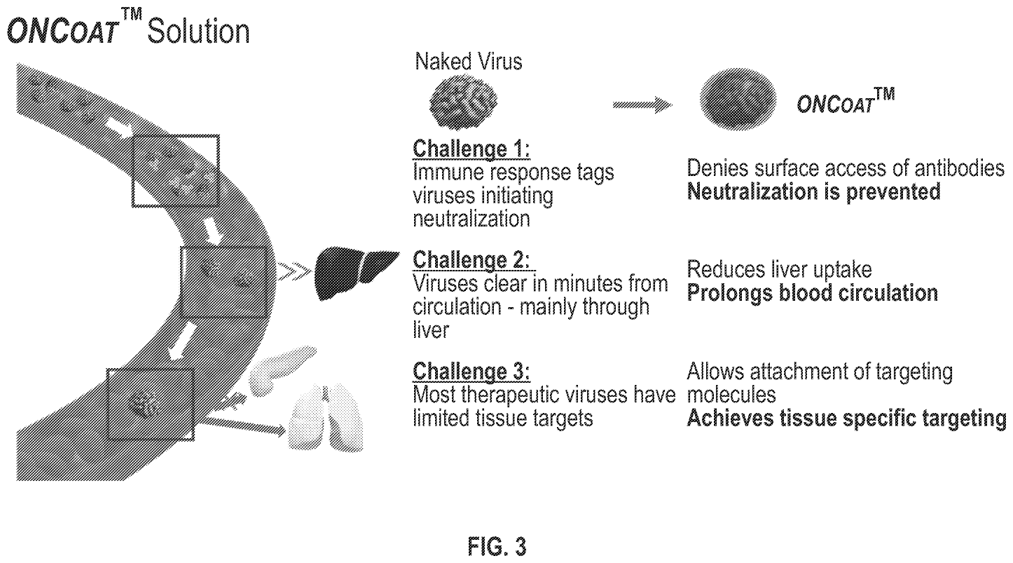

The surface of the surface-engineered recombinant virus or viral genome can be readily functionalized with ligands and other molecules to impart new properties to the surface-engineered recombinant virus or viral genome. In an embodiment, the functional molecule is attached to the surface of the surface-engineered recombinant virus or viral genome using a bifunctional spacer molecule with an anchor at one end for anchoring to the surface of the surface-engineered recombinant virus or viral genome, and a functional molecule such as a ligand at the other end. In an embodiment, the spacer is PEG, and the anchor is silane. One end of the PEG is functionalized with silane and the other is functionalized with the ligand. For attachment to the surface of the surface-engineered recombinant virus or viral genome, the silane in the ligand-PEG-silane molecule is hydrolyzed, and the ligand-PEG-silane molecule is then mixed with the surface-engineered recombinant virus or viral genome. The hydrolyzed silane attaches to the coating material on the surface of the surface-engineered recombinant virus or viral genome, such that it anchors the ligand-PEG-silane molecule to the surface of the surface-engineered recombinant virus or viral genome and displays the ligand on the surface. This functionalization imparts the properties of the ligand to the surface-engineered recombinant virus or viral genome. For example, in an embodiment where the ligand is folate, attachment of folate-PEG-silane to the surface of the surface-engineered recombinant virus or viral genome allows targeting of cancer cells. FIG. 3 shows the benefits provided by the surface engineering, including preventing neutralization, reducing liver uptake and clearance, and improving tissue targeting.

The use of silane chemistry for attachment of ligands and other functional molecules to the surface of the surface-engineered recombinant virus or viral genome is advantageous because it is simple and easily performed, and silane provides easy and biocompatible attachment that is stable in physiological conditions. The attachment reaction can be performed in physiological buffered conditions in a matter of minutes to a few hours with very high density, without negatively affecting the biological activity of the payload or virus. The ease of the attachment reaction also allows easy interchangeability of ligands during manufacturing; stocks of different ligand-PEG-silane molecules can be kept and used to impart different properties to the surface-engineered recombinant virus or viral genome as needed.

In addition to silane, any anchor molecule or moiety reasonably expected to attach to the coating material can be used to anchor the functional molecule to the surface of the surface-engineered recombinant virus or viral genome. Such an anchor can attach to the coating material, and thereby attach to the surface of the surface-engineered recombinant virus or viral genome, using ionic bonds, electrostatic interactions, covalent bonds, hydrogen bonds, van der Waals interactions, metallic bonds, physical interactions, precipitation, or any other kind of bond or interaction known by those of skill in the art to attach molecules to the materials used in the coating. Combinations of different anchors can be used. Such anchors and their use to attach molecules to substrates are well known to those of skill in the art. Such anchors include, but are not limited to, amino acids such as L-arginine and L-lysine; amine or thiol groups introduced to the silica coating; and siloxanes, silanols, alkoxysilanes, aminosilanes, or chlorosilanes. These anchors are commonly used to functionalize silica and their use is known to those of skill in the art; other anchors known in the art to functionalize silica can also be used when the coating is silica. Additionally, any reaction capable of reacting with free silyl hydroxide moieties present or introduced in the surface of the silica coating may be used to covalently modify the surface. For example, the surface of the silica coating may be treated with a trialkoxysilyl compound or trihydroxysilyl compound. The compound reacts with the silyl hydroxide surface of the silica body, forming covalent silicon-oxygen bonds. Trialkoxysilyl and trihydroxysilyl compounds may be used to modify the surface of the silica coating. The anchor can be trihydroxysilyl propyl methylphosphonate. Anchors for covalent bonding can be amine-NETS (N-hydroxysuccinimide), carboxylate-1-ethyl-3-(3-dimethylamonipropyl) carbodiimide (EDC)-amine, carboxylate-EDC+NHS-amine, amine/sulfhydryl-epoxide, amine-isothiocyanate, amine-azlactone, amine-p-nitrophenyl ester, amine-tyrosinase (TR)-tyrosine, sulfhydryl-maleimide, reactive hydrogen-benzophenone. These covalent anchors can also be used when amine groups are introduced into the silica coating. An enormous variety of covalent conjugation chemistries beyond those listed here are known to those of skill in the art. See, for example Kim et al. Biomicrofluidics 7, 041501 (2013), Rusmini et al. Biomacromolecules 8, 1775 (2007), and Hermansson Bioconjugate Techniques, 2nd ed. (Academic Press, San Diego, 2008), all incorporated herein by reference. These types of covalent conjugation chemistries can also be used to conjugate the functional molecule to the PEG.

The PEG used as a spacer molecule to connect the silane anchor with the functional molecule can be of any molecular weight appropriate for PEG spacers used to functionalize nanoparticles or microparticles. The PEG can have a molecular weight of 200 Da to 30 KDa or more. The PEG can have a molecular weight of 5 KDa, 10 KDa, or 20 KDa. The attachment of the PEG to the surface of the surface-engineered recombinant virus or viral genome using the anchor results in a PEG layer over the surface that provides additional protection from neutralization by antibodies as well as from opsonization. This PEG layer supplements the inventive coating's functions of preventing antibodies and other proteins or macromolecules from accessing the surface of the recombinant virus and preventing immune recognition of the virus. The protection provided by the PEG layer and inventive coating also improves the stability in serum/blood conditions and provides longer circulation and effective tissue accumulation. As is well known to those of skill in the art, tissue accumulation can be enhanced by the EPR effect, particularly in cancer. By using bifunctional PEG as a spacer in this manner, a single molecule with both stability and targeting capability can be obtained with a single-step reaction.

As an alternative to PEG, any spacer molecule used by those of skill in the art, including any bifunctional spacer used to connect ligands and substrates, can be used. Such spacer molecules are well known to those of skill in the art. The spacer can be a C1 to C12 alkyl chain. In other words, a C1 to C12 alkyl group is present between the atom covalently bonded to the surface of the coating and the functional molecule to be anchored to the surface of the coating. In other embodiments, the functional molecule is covalently bonded to the silica surface via a C1 to C6 alkyl linker. As used herein a C1 to C12 alkyl chain includes linear, branched and cyclic structures having 1 to 12 carbon atoms, and hybrids thereof, such as cycloalkylalkyl. Examples of alkyl chains include methylene (CH2), ethylene (CH2CH2), propylene (CH2CH2CH2), and so forth. The spacer molecule can be a chain formed from dialdehyde molecules, anhydride molecules, dichloride molecules, epihalohydrin molecules, or diepoxide molecules. The spacer can be epichlorohydrin, epibromohydrin, or epifluorohydrin. The spacer can be a water-soluble polymer, a nucleic acid, a polypeptide, an oligosaccharide, a carbohydrate, a lipid, or an ethylglycol. Combinations of different spacer molecules can be used, or the spacer molecule can be excluded, with the ligand directly attached to the anchor material anchoring the ligand to the coating over the recombinant virus or viral genome.

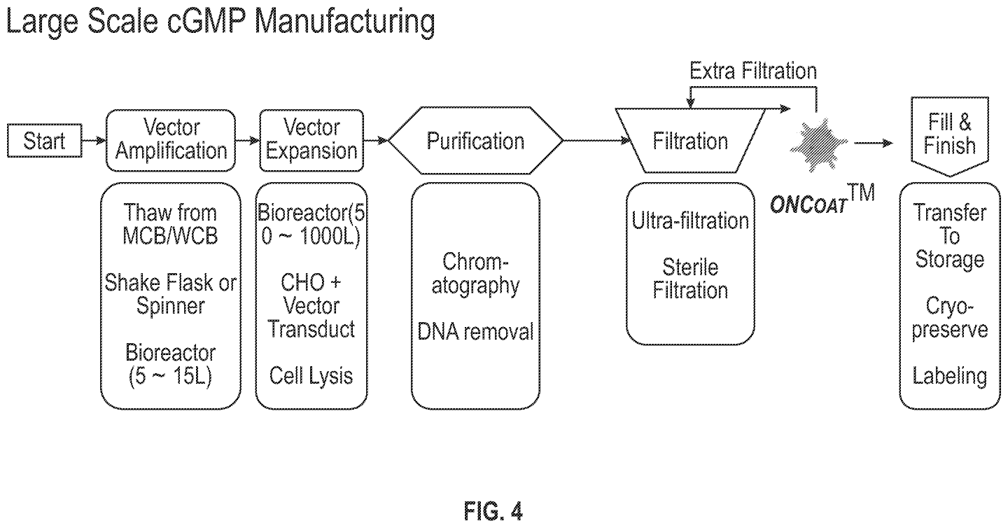

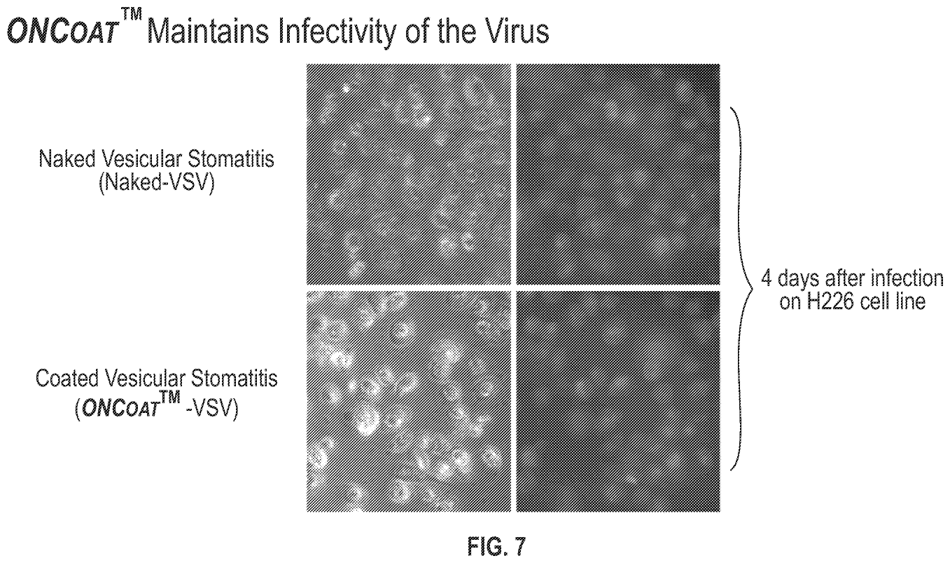

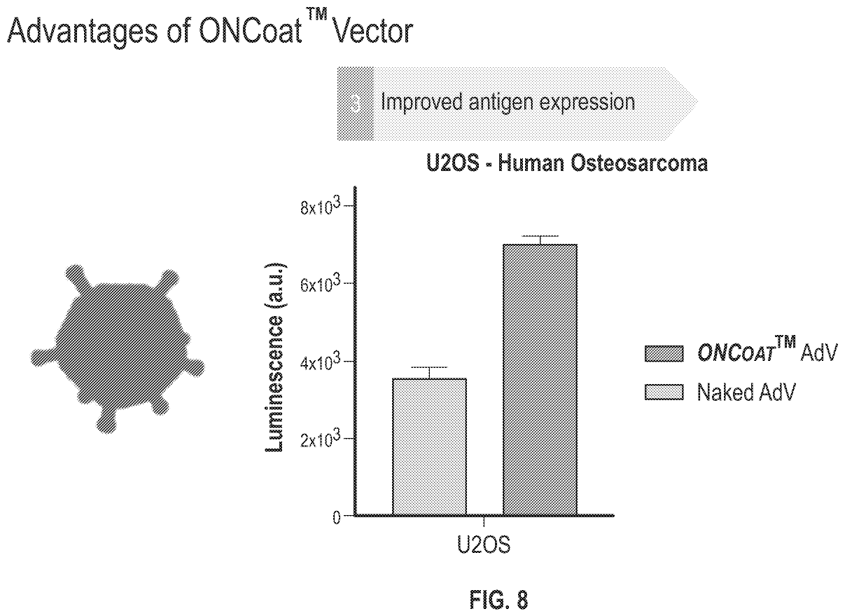

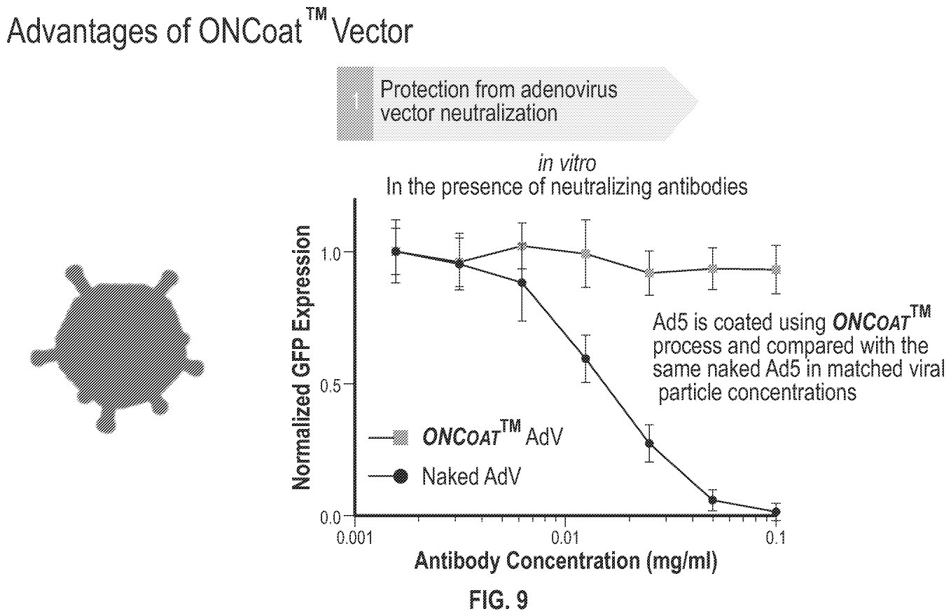

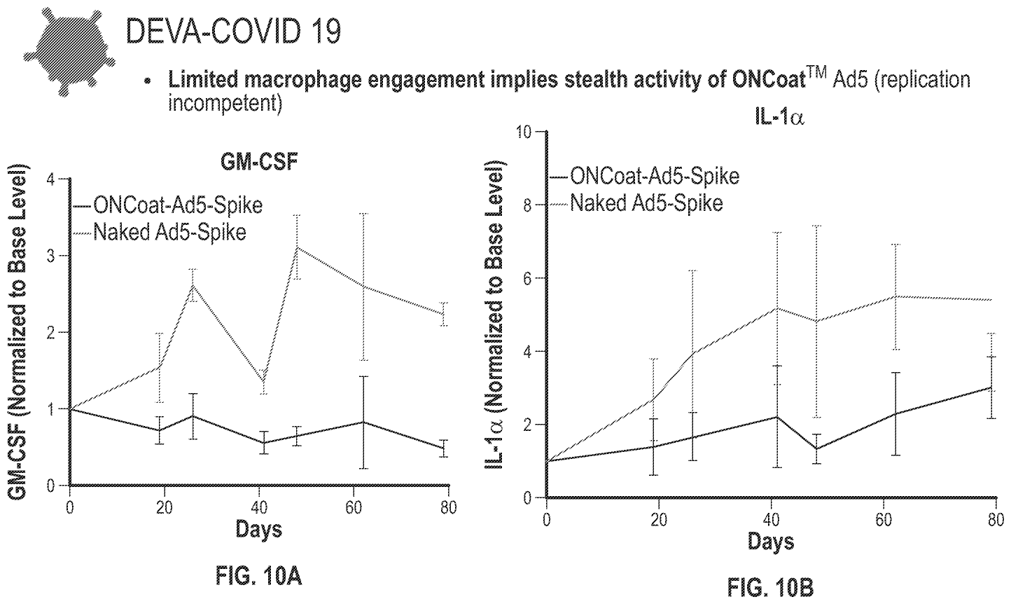

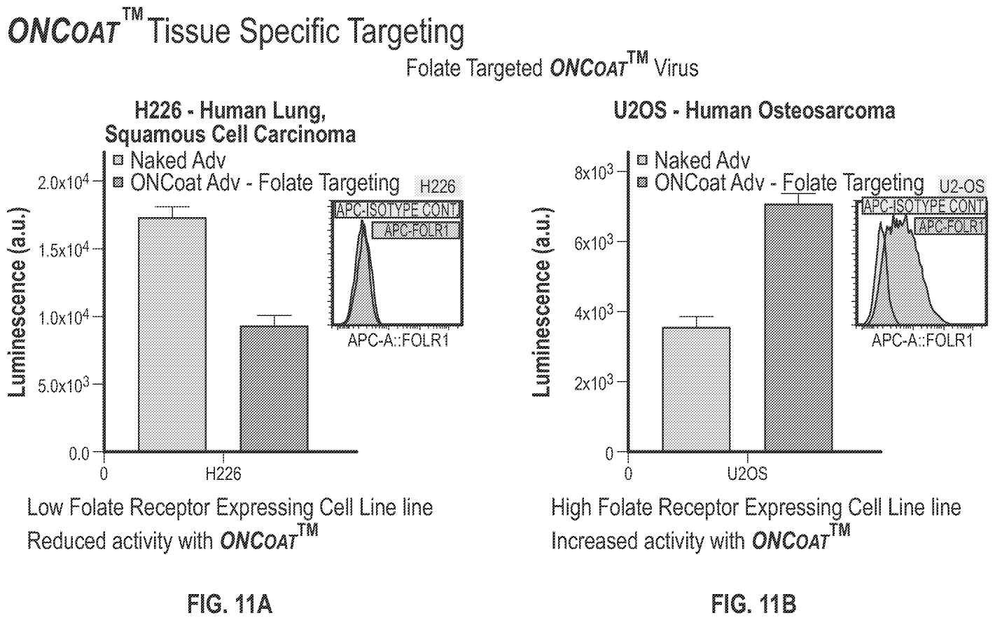

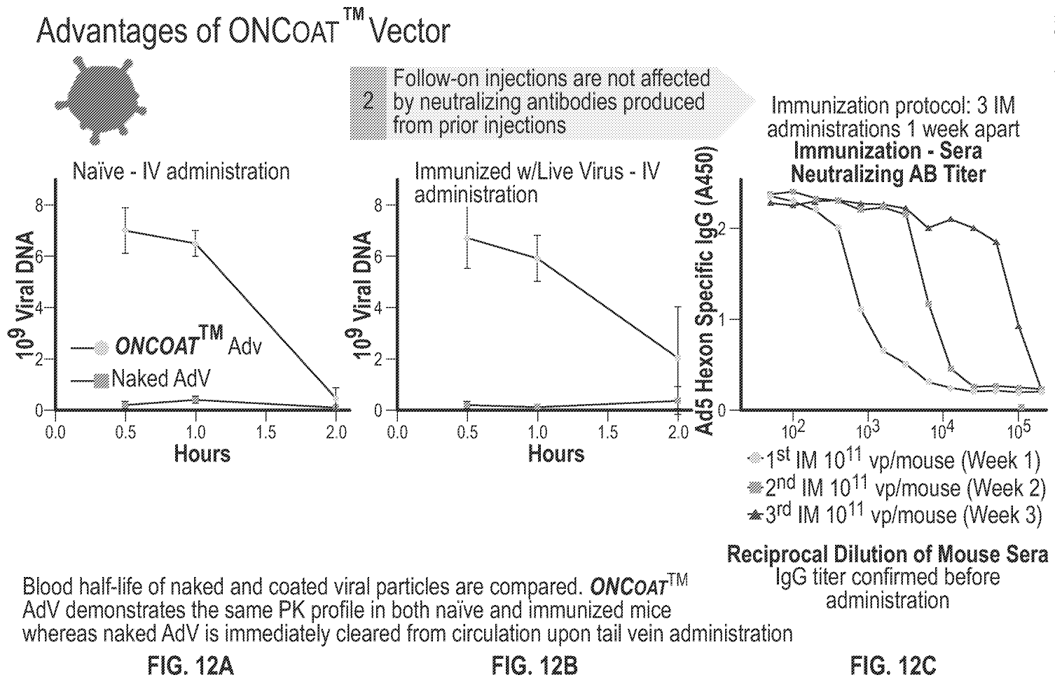

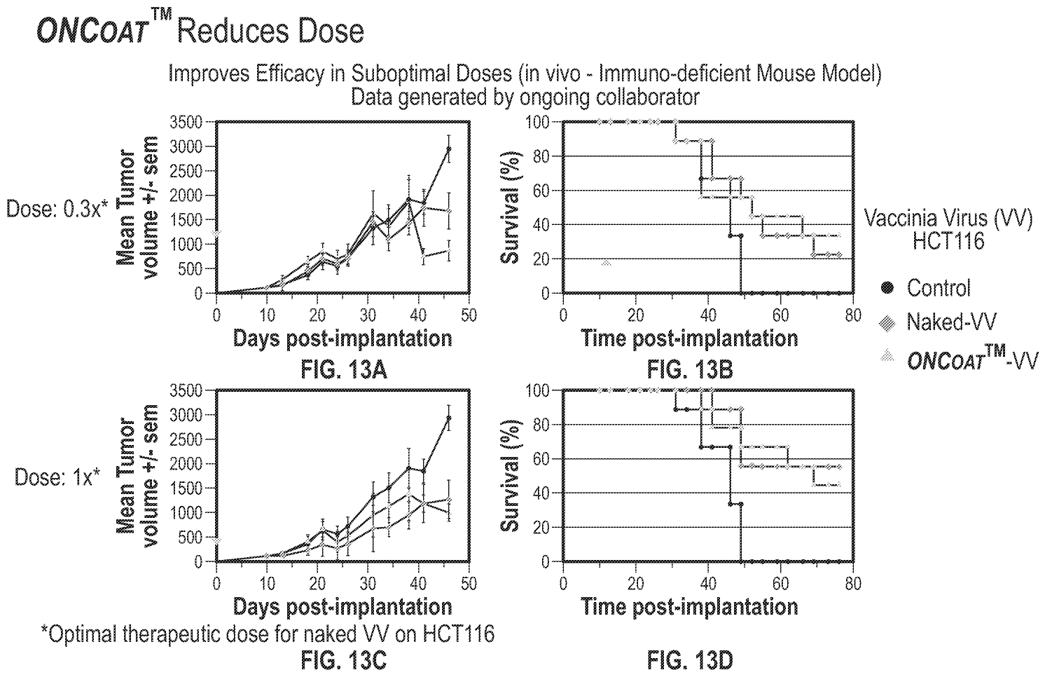

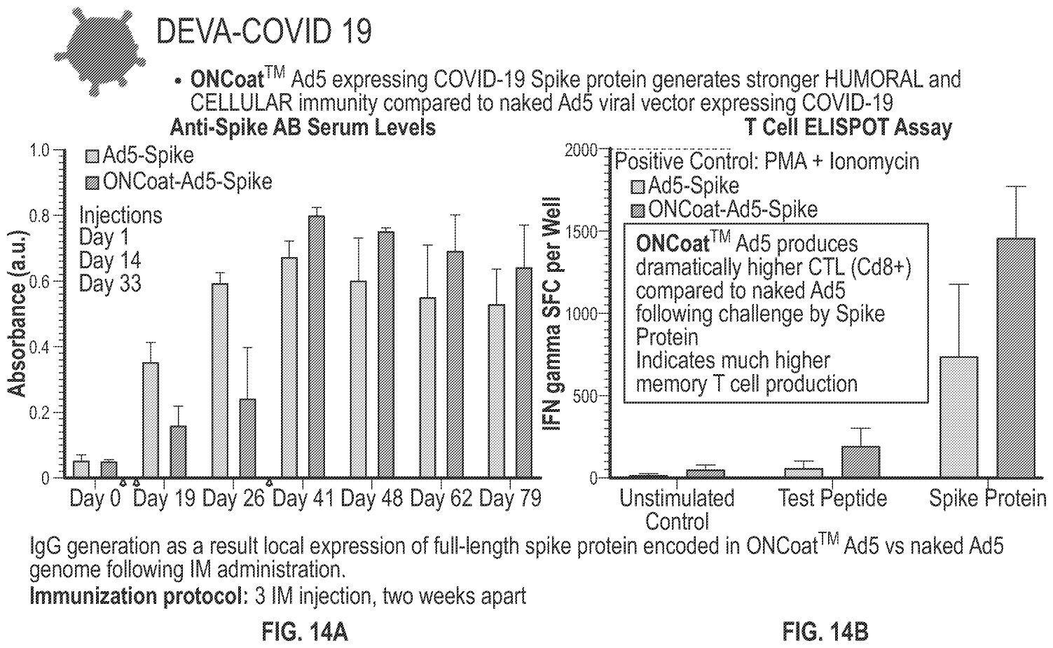

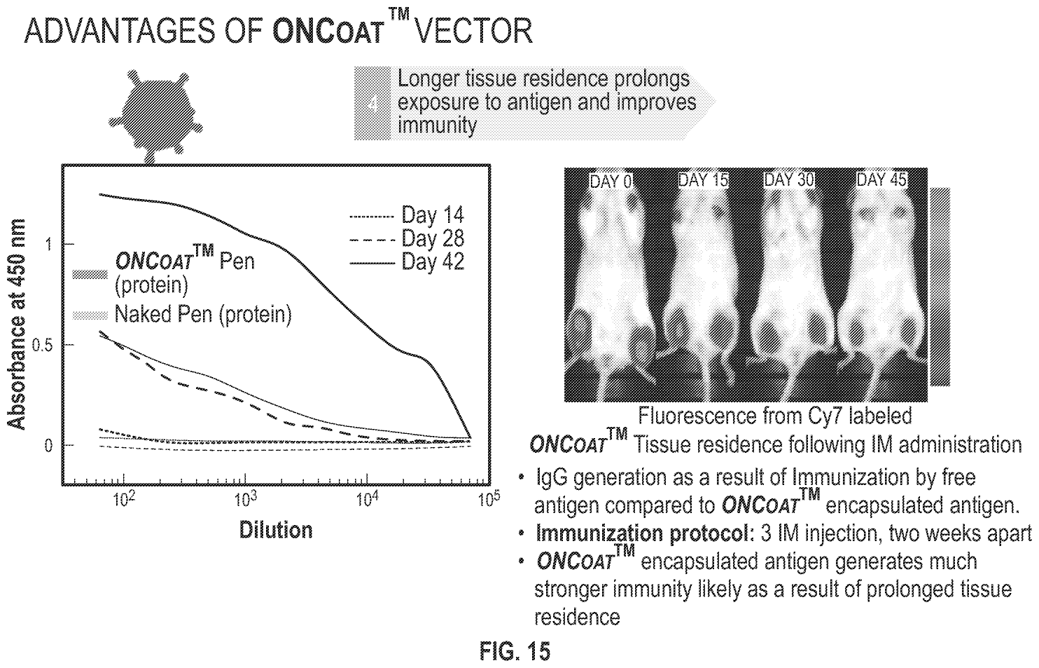

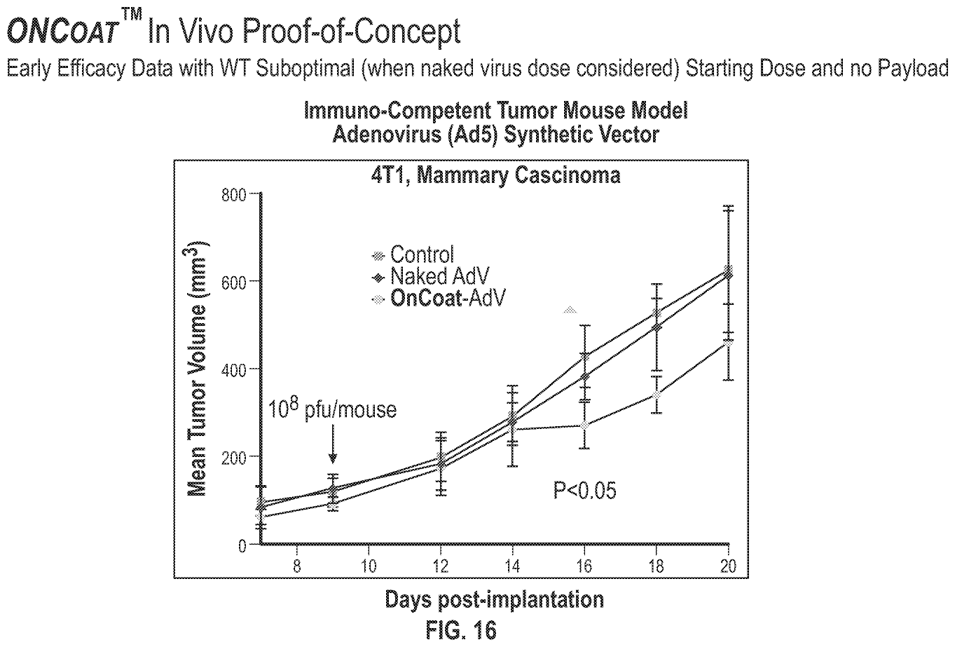

The self-assembling and self-terminating nature of the coating and ligand-anchoring processes, together with the fact that these processes are performed at room temperature and neutral pH in one-step reactions in bulk in aqueous conditions in a matter of minutes to hours, means that these processes can be easily scaled for large-scale GMP-grade manufacturing. The manufacturing scheme is shown in FIG. 4. The gentle and inert reaction conditions and nature of the reaction mean that the coating and ligand-anchoring processes do not damage the virus or negatively affect its infectivity. FIG. 7 shows that the coating does not negatively affect the infectivity.

The coating, with or without attached PEG, prevents or reduces toxicity of the recombinant virus upon administration. Lack of toxicity can be determined by normal levels of biomarkers where abnormal levels would be indicative of toxicity. Such biomarkers are well known to those of ordinary skill in the art. Lack of liver toxicity can be determined by normal aspartate transaminase (AST) and normal alanine transaminase (ALT) activity. In experiments conducted by the inventor, after three injections of 5×106 pfu/mouse (injections 2 days apart, livers harvested right after last injection), AST activity was 29+/−5 U/L (Normal: 25-100 U/L) and ALT activity was 27+/−12 U/L (Normal: 25-60 U/L).

The coating, with or without attached PEG, provides a “stealth” property such that it prevents or reduces recognition by macrophages and/or activation of macrophages. This helps to increase the circulation time and permit systemic administration and repeated administration. It may also help to reduce toxicity.

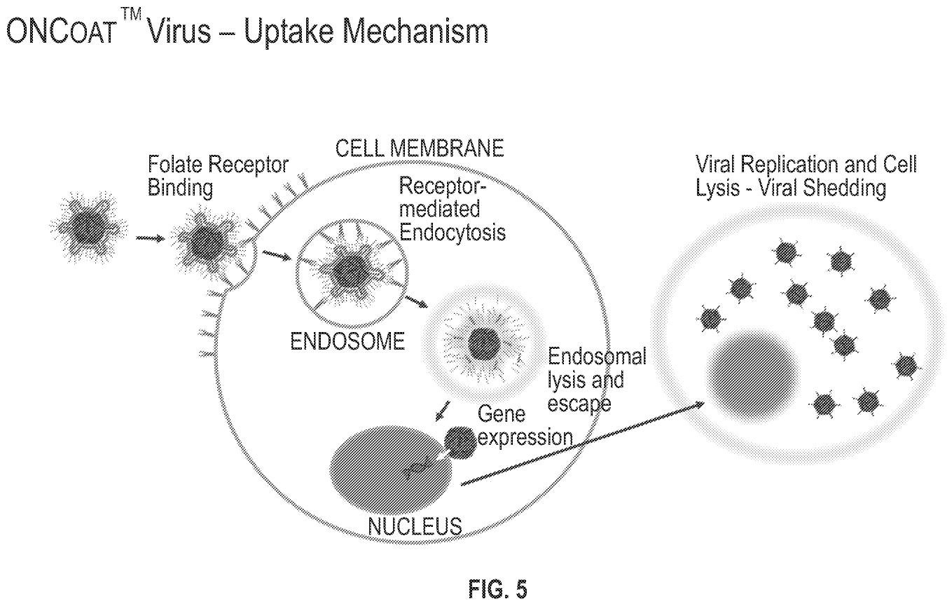

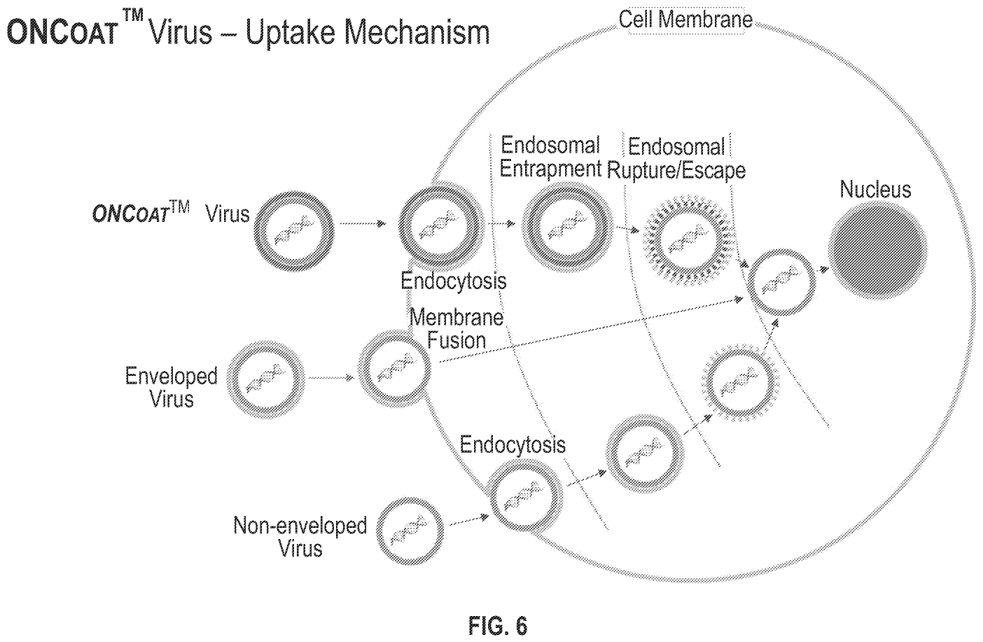

Folate is an exemplary functional molecule used to allow targeting of the surface-engineered recombinant virus or viral genome to cancer cells, such that the folate is attached to the coating over the surface-engineered recombinant virus or viral genome using a silane anchor with a PEG spacer. Folate-functionalized surface-engineered recombinant virus or viral genome can enter cells via receptor-mediated uptake, followed by accumulation in endosomes. In particular, in the late endosomal stage, when the pH inside the endosome decreases to approximately pH 5, the coating on the surface-engineered recombinant virus or viral genome degrades, releasing silicic acid and revealing the cationic polymer on the virus surface. This process eventually leads to rupture of the endosome, releasing the viral capsid into the cytoplasm. In another embodiment, the viral capsid is released in the endosome, escaping the endosome using its own machinery based on features of the capsid/core. When the viral capsid is released into the cytoplasm it can reach the nucleus and release its genomic payload into the nucleus using its natural processes as they are not negatively affected by the coating process used for surface engineering. In the case of RNA viruses, release into the cytoplasm will trigger expression of the genetic payload without need for delivering the payload into the nucleus. The coating with surface functionalization provides a shuttle for the recombinant virus payload through the circulation and targeted tissue into the cell. Inside the cell, the degradation of the coating allows the virus to complete its life cycle effectively. The uptake mechanism in shown in FIG. 5 and FIG. 6.

In addition to folate, any molecule known to have a relevant function can be similarly attached to the surface of the surface-engineered recombinant virus or viral genome, i.e., to the coating. For example, such molecules can allow targeting of the surface-engineered recombinant virus or viral genome to a particular cell, tissue, or organ. Such molecules can allow the surface-engineered recombinant virus or viral genome to cross biological barriers, such as blood vessel walls or the blood-brain barrier. Such molecules can facilitate uptake of the surface-engineered recombinant virus or viral genome into cells. Such molecules can induce endosomolysis or allow intracellular trafficking of the surface-engineered recombinant virus or viral genome to particular intracellular organelles such as the nucleus or mitochondria. Such molecules can allow the surface-engineered recombinant virus or viral genome to attach to a substrate such a biomaterial matrix to allow controlled release of the surface-engineered recombinant virus or viral genome. Such molecules can allow the surface-engineered recombinant virus or viral genome to attach to a substrate such as an affinity column or magnetic beads for isolation or purification. Such molecules can provide an immunostimulatory or immunomodulatory effect. Such molecules can have a pharmaceutical effect, for example blocking ion channels. Such molecules can be magnetic or susceptible to magnetic fields, such that a magnetic field can then be used to guide the surface-engineered recombinant virus or viral genome to a desired place in the body. Such molecules are well known to those of skill in the art. Such molecules can be proteins, peptides, polysaccharides, carbohydrates, lipids, fatty acids, synthetic or natural polymers, small molecules, RNA, DNA, aptamers, antibodies, antibody fragments, antigens, epitopes, cytokines, fluorescent markers, and/or growth factors. Examples of such functional molecules include, but are not limited to, antibodies or ligands that target the estrogen receptor, epidermal growth factor receptor, CD47, HER2 receptor, IL-4 receptor, AXL, ALK, PTK7, TM4F1, nectin4, PSMA, VEGFR, CTLA4, ERB22, CD20, CD22, CD30, CD33, CD52, CD74, CD276, and/or CD79. Such functional molecules also include TAT peptide, SV40 large T antigen, nuclear localization signal (NLS) peptides, cell penetrating peptides, and/or endosomolytic peptides such as melittin. Such functional molecules also include PECAM, transferrin, melanotransferrin, alanine, glutathione, OX26, and antibodies and ligands targeting GLUT-1, LRP1, TfR1, and/or SLC7A5 receptor. Such functional molecules also include hyaluronic acid, RGD peptide, Tyr3-octreotide, PE-221, octreotide, rabies virus glycoprotein-29, miniAp-4, angiopep-1, iRGD (CEND-1), BT1718, PL1, CARG, cyclic RGD peptide, IL4RPep-1, AS-1411 aptamer, anti-VEGF aptamer, A-9 aptamer, A-10 aptamer, anti-gp120 aptamer, TTA 1 aptamer, sgc8 aptamer, anti-MUC-1 aptamer, GBI-10 aptamer, anisamide, phenylboronic acid, folic acid, glucose, galactose, glutamate urea, vitamin A, mannose, and biotin. Such functional molecules include glycyrrhetinic acid, sulfonamide, and derivatives thereof.

Different functional molecules with similar or different functions can be used in combination to simultaneously impart multiple different functions and capabilities to the surface-engineered recombinant virus or viral genome. For example, two different cell-targeting ligands can be used together to increase the specificity of cell targeting. As another example, a cell-targeting ligand is used with an uptake-enhancing ligand to allow the surface-engineered recombinant virus or viral genome to both target a particular cell type and then to facilitate entry into the cell. In embodiments where different such molecules are used, each molecule is attached to a linker-spacer material such as silane-PEG as discussed above, and then the different molecule-PEG-silane conjugates are mixed together with the surface-engineered recombinant virus or viral genome to allow the different molecules to simultaneously attach to the surface-engineered recombinant virus or viral genome in the desired stoichiometry. The molecules and their associated functions are thus easily interchangeable during manufacturing.

As set forth herein, the surface can be further functionalized with stabilization and targeting ligands. In one particular embodiment, polyethylene glycol (PEG) is used to functionalize a silane group at one end and folate in the other hand. In particular embodiments, PEG can be a monomer, or PEG lengths can be selected from 1, 2, 3, 4, 5, 6, 7, 8, 9, 10, 11, 12, 13, 14, 15, 16, 17, 18, 19, or 20 KDa. PEG silane is added into the solution, and the silane group is hydrolyzed and attaches to the silica surface. pH-responsive linkers known to those of skill in the art can be used such that the PEG is stripped from the surface-engineered delivery system under conditions with altered pH, such as in the vicinity of tumors.

In certain embodiments, the PEG polymer is dual functionalized. On one end, it can be functionalized with silane and on the other end it can be functionalized with a targeting ligand like folate. Silane is used to attached the polymer to the ONCoat surface while folate can be utilized to target to the tissues and trigger cellular uptake, while PEG is being utilized to prevent neutralization and improve the pharmacokinetics. Targeting ligands include carbohydrates (e.g. galactose), monoclonal antibodies (e.g., anti-Her2, anti-EGFR), peptides (e.g., Arg-Gly-Asp or RGD), proteins (e.g., lectins, transferrin), vitamins (e.g., Vitamin D), and aptamers (e.g., RNA aptamers against HIV glycoprotein) and agonists/antagonists for toll-like receptors (TLR) such as TLR1, TLR2, TLR4, TLR7, TLR8, and TLR9 the like.

In certain other embodiments of the invention provided herein, the coating-layer further comprises molecules (e.g., binding agents such as targeting-ligands) on its surface that change the cellular uptake and/or biological activity of surface-engineered delivery system, for example the uptake or activity of the recombinant virus relative to the native-virus or the uptake or activity of the nucleic acid relative to the naked nucleic acid. Thus, the infectivity and host range of the surface-engineered or re-engineered virus can be selected and/or controlled by selecting and incorporating the appropriate targeting-ligands on the outer surface of the OnCoat artificial coating layer. The infectivity can thereby be increased. Additionally, this can reduce the dose required to obtain efficacy, and allow efficacy to be obtained at lower doses. When the virus is a vaccine, OnCoat increases the humoral immunity (antibody levels) and cellular immunity (T lymphocyte levels) produced by the vaccine. When the virus is an anti-cancer virus, OnCoat increases the anti-tumor efficacy. In particular embodiments of the surface-engineered delivery system, the artificial coating comprises a cell-surface ligand selected from the group consisting of: proteins, polysaccharides, aptamers, peptides, oligonucleotides and small molecules. In some embodiments, the artificial coating comprises a cell-penetrating molecule, such as a cell-penetrating peptide such as TAT, CADY, TP, or TP10. In some embodiments, the artificial coating comprises a targeting-ligand selected from the group consisting of: Antibodies, transferrin, Hyaluronic acid, RGD (e.g., cRGD), IL4RPep-1, AS-1411, GBI-10, Folate, anisamide, and phenylboronic acid. In particular embodiments, folate is used in the coating layer as ligand for binding to cells with folate receptors, such as cancer cells, and the like. In particular embodiments, antibodies against CD47 are used in the coating layer as ligand for binding to cells with surface expression of CD47, such as cancer cells, and the like. In some embodiments, the targeting-ligand is one that enables the surface-engineered delivery system to cross the blood-brain barrier, for example mannose or other glucose transporter ligands or binding molecules. Multiple different targeting-ligands can be combined to provide increased targeting specificity. Multiple different types of molecules in the coating-layer can be combined to provide multifunctionality, for example combining a targeting-ligand with a cell-penetrating molecule to enable the surface-engineered delivery system to both target and then enter a particular cell type.

In other embodiments, of the invention surface-engineered virus, the recombinant virus can be replication-competent or replication-defective, or “gutted” such that the virus does not encode any viral proteins. As used herein, the phrase “replication-defective virus” refers to a virus that is specifically defective for viral functions that are essential for viral genome replication and assembly of progeny virus particles. They are propagated in complementing cell lines that express the missing viral gene product(s), allowing viral replication to produce a stock of replication-defective virus.

Also provided herein is a surface-engineered recombinant virus vaccine, said virus comprising:
a recombinant Ad5 virus having a recombinant genome encoding SARS-CoV-2 spike protein, wherein at least a functional portion of E1 and E3 genes are deleted; and an artificial coating layer encapsulating the recombinant Ad5 virus. In a particular embodiment, as set forth herein, the artificial coating layer comprises an effective amount of ligand to bind a ligand receptor on a cell. In another embodiment, the artificial coating is selected from the group consisting of: silica, titanium oxide and calcium phosphate.

As used herein, the phrase “oncolytic virus” refers to a virus that preferentially infects and kills cancer cells. As the infected cancer cells are destroyed by oncolysis, they release new infectious virus particles or virions to help destroy the remaining tumor or tumors.

As used herein, the phrase “SARS-Cov-2 spike protein” refers to the glycoprotein on SARS-CoV-2 that promotes entry into cells using the ACE2 cell-surface receptor to enter cells. See, e.g., Walls et al., 2020, Cell, 180, 281-292 (which is incorporated herein by reference in its entirety for all purposes) for the spike protein sequence. Either the full-length spike protein sequence or any fragment thereof is contemplated for use herein with the invention artificially coated viral vaccines.

As used herein, the phrase “Ad5 virus” refers to the adenovirus serotype 5 that have been repeatedly used in humans to induce robust T cell-mediated immune (CMI) responses, all while maintaining an extensive safety profile (see, e.g, U.S. Pat. No. 9,605,276, and the like). Ad5 vectors can be reliably manufactured in large quantities and are stable for storage and delivery for outpatient administration.

In certain embodiments, the adenovirus vectors contemplated for use in the present invention include E1 and E3 deleted adenovirus vectors that have a deletion in the E1 and E3 region of the Ad genome and, optionally, the E2b region. In some cases, such vectors do not have any other regions of the Ad genome deleted. In another embodiment, the adenovirus vectors contemplated for use in the present invention include E1 deleted adenovirus vectors that have a deletion in the E1 region of the Ad genome and, optionally, deletions in the E3, E4 and/or E2b regions. In some cases, such vectors have no other regions deleted. In another embodiment, the adenovirus vectors contemplated for use in the present invention include E3 deleted adenovirus vectors that have a deletion in the E3 region of the Ad genome and, optionally, deletions in the E1, E4 and/or E2b regions. In some cases, such vectors have no other regions deleted. In a further embodiment, the adenovirus vectors contemplated for use in the present invention include adenovirus vectors that have a deletion in the E1 and E3 regions of the Ad genome and, optionally, deletions in the E2b and/or partial or complete removal of the E4 regions. In some cases, such vectors have no other deletions. In an additional embodiment, the adenovirus vectors contemplated for use in the present invention include adenovirus vectors that have a deletion in the E2a, E2b and/or E4 regions of the Ad genome. In some cases, such vectors have no other deletions. In one embodiment, the adenovirus vectors for use herein comprise vectors having the E1 and/or DNA polymerase functions of the E2b region deleted. In some cases, such vectors have no other deletions. In a further embodiment, the adenovirus vectors for use herein have the E1 and/or the preterminal protein functions of the E2b region deleted. In some cases, such vectors have no other deletions. In another embodiment, the adenovirus vectors for use herein have the E1, DNA polymerase and/or the preterminal protein functions deleted. In some cases, such vectors have no other deletions. In one particular embodiment, the adenovirus vectors contemplated for use herein have at least a portion of the E2b region and/or the E1 region deleted. In some cases, such vectors are not “gutted” adenovirus vectors. In this regard, the vectors may have both the DNA polymerase and the preterminal protein functions of the E2b region deleted. In an additional embodiment, the adenovirus vectors for use in the present invention include adenovirus vectors that have a deletion in the E1, E2b and/or 100K regions of the adenovirus genome. In one embodiment, the adenovirus vectors for use herein comprise vectors having the E1, E2b and/or protease functions deleted. In some cases, such vectors have no other deletions. In a further embodiment, the adenovirus vectors for use herein have the E1 and/or the E2b regions deleted, while the fiber genes have been modified by mutation or other alterations (for example to alter Ad tropism). Removal of genes from the E3 or E4 regions may be added to any of the mentioned adenovirus vectors. In certain embodiments, the adenovirus vector may be a “gutted” adenovirus vector.

In certain embodiments, the virus can express additional cytokines (e.g., at least one subunit of IL12, GM-CSF and the like) or/and chemokines together with the at least one subunit of Spike protein to enhance improve the tumor engagement.

As used herein, the phrase “E1 deleted” or “a functional portion of the E1 gene is deleted,” or grammatical variations thereof, refers to a specific DNA sequence that is mutated in such a way so as to prevent expression and/or function of at least one E1 gene product. Thus, in certain embodiments, “E1 deleted” is used in relation to a specific DNA sequence that is deleted (removed) from the Ad genome. E1 deleted or “containing a deletion within the E1 region” refers to a deletion of at least one base pair within the E1 region of the Ad genome. Thus, in certain embodiments, more than one base pair is deleted and in further embodiments, at least 20, 30, 40, 50, 60, 70, 80, 90, 100, 110, 120, 130, 140, or 150 base pairs are deleted. In another embodiment, the deletion is of more than 150, 160, 170, 180, 190, 200, 250, or 300 base pairs within the E1 region of the Ad genome. An E1 deletion may be a deletion that prevents expression and/or function of at least one E1 gene product and therefore, encompasses deletions within exons of encoding portions of E1-specific proteins as well as deletions within promoter and leader sequences. In certain embodiments, an E1 deletion is a deletion that prevents expression and/or function of one or both of a trans-acting transcriptional regulatory factor of the E1 region. In a further embodiment, “E1 deleted” refers to one or more point mutations in the DNA sequence of this region of an Ad genome such that one or more encoded proteins is non-functional. Such mutations include residues that are replaced with a different residue leading to a change in the amino acid sequence that result in a nonfunctional protein.

As used herein, the phrase “E3 deleted” or “a functional portion of the E3 gene is deleted,” or grammatical variations thereof, refers to a specific DNA sequence that is mutated in such a way so as to prevent expression and/or function of at least one E3 gene product. Thus, in certain embodiments, “E3 deleted” is used in relation to a specific DNA sequence that is deleted (removed) from the Ad genome. E3 deleted or “containing a deletion within the E3 region” refers to a deletion of at least one base pair within the E3 region of the Ad genome. Thus, in certain embodiments, more than one base pair is deleted and in further embodiments, at least 20, 30, 40, 50, 60, 70, 80, 90, 100, 110, 120, 130, 140, or 150 base pairs are deleted. In another embodiment, the deletion is of more than 150, 160, 170, 180, 190, 200, 250, or 300 base pairs within the E3 region of the Ad genome. An E3 deletion may be a deletion that prevents expression and/or function of at least one E3 gene product within the expression cassette and therefore, encompasses deletions within exons of encoding portions of E3-specific proteins as well as deletions within promoter and leader sequences. In certain embodiments, an E3 deletion is a deletion that prevents expression and/or function of at least one of the host immune response modulating proteins of the E3 region (see, e.g., Arnberg, PNAS, 2013, 110(50):19976-19977). In a further embodiment, “E3 deleted” refers to one or more point mutations in the DNA sequence of this region of an Ad genome such that one or more encoded proteins is non-functional. Such mutations include residues that are replaced with a different residue leading to a change in the amino acid sequence that result in a nonfunctional protein.

As used herein, the phrase “E2b deleted” or “a functional portion of the E2b gene is deleted,” or grammatical variations thereof, refers to a specific DNA sequence that is mutated in such a way so as to prevent expression and/or function of at least one E2b gene product. Thus, in certain embodiments, “E2b deleted” is used in relation to a specific DNA sequence that is deleted (removed) from the Ad genome. E2b deleted or “containing a deletion within the E2b region” refers to a deletion of at least one base pair within the E2b region of the Ad genome. Thus, in certain embodiments, more than one base pair is deleted and in further embodiments, at least 20, 30, 40, 50, 60, 70, 80, 90, 100, 110, 120, 130, 140, or 150 base pairs are deleted. In another embodiment, the deletion is of more than 150, 160, 170, 180, 190, 200, 250, or 300 base pairs within the E2b region of the Ad genome. An E2b deletion may be a deletion that prevents expression and/or function of at least one E2b gene product and therefore, encompasses deletions within exons of encoding portions of E2b-specific proteins as well as deletions within promoter and leader sequences. In certain embodiments, an E2b deletion is a deletion that prevents expression and/or function of one or both of the DNA polymerase and the preterminal protein of the E2b region. In a further embodiment, “E2b deleted” refers to one or more point mutations in the DNA sequence of this region of an Ad genome such that one or more encoded proteins is non-functional. Such mutations include residues that are replaced with a different residue leading to a change in the amino acid sequence that result in a nonfunctional protein.

As would be understood by the skilled artisan upon reading the present disclosure, other regions of the Ad genome can be deleted. Thus to be “deleted” in a particular region of the Ad genome, as used herein, refers to a specific DNA sequence that is mutated in such a way so as to prevent expression and/or function of at least one gene product encoded by that region. In certain embodiments, to be “deleted” in a particular region refers to a specific DNA sequence that is deleted (removed) from the Ad genome in such a way so as to prevent the expression and/or the function encoded by that region (e.g., E2b functions of DNA polymerase or preterminal protein function). “Deleted” or “containing a deletion” within a particular region refers to a deletion of at least one base pair within that region of the Ad genome. Thus, in certain embodiments, more than one base pair is deleted and in further embodiments, at least 20, 30, 40, 50, 60, 70, 80, 90, 100, 110, 120, 130, 140, or 150 base pairs are deleted from a particular region. In another embodiment, the deletion is more than 150, 160, 170, 180, 190, 200, 250, or 300 base pairs within a particular region of the Ad genome. These deletions are such that expression and/or function of the gene product encoded by the region is prevented. Thus deletions encompass deletions within exons encoding portions of proteins as well as deletions within promoter and leader sequences. In a further embodiment, “deleted” in a particular region of the Ad genome refers to one or more point mutations in the DNA sequence of this region of an Ad genome such that one or more encoded proteins is non-functional. Such mutations include residues that are replaced with a different residue leading to a change in the amino acid sequence that result in a nonfunctional protein. Deletions or mutations in the Ad genome can be within one or more of E1a, E1b, E2a, E2b, E3, E4, L1, L2, L3, L4, L5, TP, POL, IV, and VA regions.

The deleted adenovirus vectors of the present invention can be generated using recombinant techniques known in the art (see e.g., Amalfitano et al., 1998 J. Virol. 72:926-933; Hodges, et al., 2000 J Gene Med 2:250-259).

Also provided herein is a method of making a surface-engineered recombinant virus, said method comprising:
producing a recombinant virus having a recombinant genome; and
applying an artificial coating layer to the recombinant virus, wherein the artificial coating is selected from the group consisting of: silica, titanium oxide and calcium phosphate.

As set forth herein, any of the viruses described herein can be genetically modified using method well-known in the art to produce a recombinant virus having a recombinant genome. The artificial coating layer can be applied to the recombinant virus as described herein.

Also provided herein, is a method of re-engineering the surface of a virus having a native-envelope, said method comprising:
removing the native-envelope from the virus to isolate a previously-enveloped-capsid; and applying an artificial coating to the previously-enveloped-capsid.

Any enveloped virus or lipid-containing virus known in the art is suitable for re-engineering using the methods provided herein.

As used herein, the phrase “re-engineering the surface of a virus having a native-envelope” refers to proactively disrupting and replacing the viral envelope (e.g., outer lipid-containing layer) of an enveloped-virus with an artificial coating layer.

As used herein, the phrase “removing the native-envelope from the virus” refers to using any means to disrupt the native outer lipid-containing layer enveloping the capsid, such that the capsid, referred to herein as the previously-enveloped-capsid, can be isolated from any remaining lipids or outer envelope layer.

As used herein, the phrase “previously-enveloped-capsid” refers to an envelope-free or naked-capsid isolated from a previously enveloped virus, such as those described herein.

The envelope of the virus can be removed using any method known in the art, including use of detergents and/or delipidation with an extraction solvent as set forth in U.S. Pat. No. 7,407,662 (which in incorporated herein in its entirety for all purposes). In particular embodiments, the native-envelope is removed or delipidated using a detergent and/or an extraction solvent. In some embodiments, the detergent and/or an extraction solvent can be selected from the group consisting of: Glutaraldehyde, chloroform, B-propiolactone, TWEEN-80, and dialkyl or trialkyl phosphates, alcohols, hydrocarbons, amines, ethers, n-butanol, di-isopropyl ether (DIPE), diethyl ether, either alone or in combination.

As used herein, the term “delipidation” refers to the process of removing at least a portion of a total concentration of lipids in a fluid or in a lipid-containing envelope of an enveloped virus described herein.

Briefly, in one embodiment, the envelope is removed and the artificial coating layer is applied as follows. The recombinant virus is incubated in aqueous buffer with 0.5-1% NP-40 (detergent) for 5 mins at room temperature. Then, the aqueous buffer is washed three times with detergent free aqueous buffer to remove the detergent and lifted envelope.

In other embodiments of the methods provided herein, a first extraction solvent is used to remove the envelope from an enveloped virus, to produce a fluid solution containing a previously-enveloped-capsid. As used herein the phrase, “first solvent” or “first organic solvent” “or first extraction solvent,” or grammatical variations thereof, refers to a solvent, comprising one or more solvents, used to facilitate extraction of lipid envelope from a lipid-containing envelope virus in the fluid. This solvent will enter the fluid and remain in the fluid until being removed. Suitable first extraction solvents include solvents that extract or dissolve lipid, including but not limited to alcohols, hydrocarbons, amines, ethers, and combinations thereof. First extraction solvents may be combinations of alcohols and ethers. First extraction solvents include, but are not limited to n-butanol, di-isopropyl ether (DIPE), diethyl ether, and combinations thereof.

In particular embodiments, second extraction solvent is used to remove the first solvent from the solution containing the de-enveloped capsid (“previously-enveloped-capsid”). As used herein the phrase “second extraction solvent” reefers to one or more solvents that may be employed to facilitate the removal of a portion of the first extraction solvent. Suitable second extraction solvents include any solvent that facilitates removal of the first extraction solvent from the fluid.

Second extraction solvents include any solvent that facilitates removal of the first extraction solvent including but not limited to ethers, alcohols, hydrocarbons, amines, and combinations thereof.

Preferred second extraction solvents include diethyl ether and di-isopropyl ether, which facilitate the removal of alcohols, such as n-butanol, from the fluid. The term “de-emulsifying agent” is a second extraction solvent that assists in the removal of the first solvent which may be present in an emulsion in an aqueous layer.

In another embodiment, the envelope on either a naturally occurring or recombinant enveloped virus is removed and replaced or artificially coated with an OnCoat coating (e.g., silica-gel coating, and the like) as described herein. See, for example, the methods described in US 2019/0151253A1 and in Ortac et al. (Nano Lett. 2014, 14, 6, 3023-3032), which are incorporated herein by reference in their entirety, for all purposes. Once the envelope is removed, then the remaining capsid can be coated with an OnCoat artificial coating layer as described herein.

As set forth herein, in certain embodiments, the artificial coating is selected from the group consisting of: silica, titanium oxide and calcium phosphate. In one embodiment, applying the artificial coating further comprises conducting a charge-mediated sol-gel condensation reaction directly onto the surface of the previously-enveloped-capsid. In another embodiment, the artificial coating comprises a silica gel matrix or titanium oxide gel matrix encapsulating the previously-enveloped-capsid. In particular embodiments, the previously-enveloped-capsid is replication-competent or replication-defective.

In particular embodiments for applying the artificial coating layer, as set forth in the methods described in US 2019/0151253A1, which is incorporated herein by reference in its entirety for all purposes, the virus is incubated with polycationic polymer to shift the virus surface charge towards the positive direction. Next, a silica precursor, such as tetramethyl (or tetraethyl) orthosilicate, and the like, is hydrolyzed to generate silicic acid, which is added into the virus suspension surface-modified with polycationic polymer. The polymer on the virus surface templates the polycondensation reaction (sol-gel) to produce silica gel, as set forth in U.S. Pat. No. 10,869,841, which is incorporated herein by reference in its entirety for all purposes.

The surface can then be further functionalized with stabilization and targeting ligands. In one particular embodiment, polyethylene glycol (PEG) is used to functionalize a silane group at one end and folate in the other hand. In particular embodiments, PEG can be a monomer, or PEG lengths can be selected from 1, 2, 3, 4, 5, 6, 7, 8, 9, 10, 11, 12, 13, 14, 15, 16, 17, 18, 19, or 20 KDa. PEG silane is added into the solution, and the silane group is hydrolyzed and attaches to the silica surface.

The infectivity and host range of the surface-engineered or re-engineered virus can be selected and/or controlled by selecting and incorporating the appropriate targeting-ligands on the outer surface of the OnCoat artificial coating layer. The infectivity can thereby be increased. Additionally, this can reduce the dose required to obtain efficacy, and allow efficacy to be obtained at lower doses. When the virus is a vaccine, OnCoat increases the humoral immunity (antibody levels) and cellular immunity (T lymphocyte levels) produced by the vaccine. When the virus is an anti-cancer virus, OnCoat increases the anti-tumor efficacy.

Any naturally occurring enveloped virus can be de-enveloped and coated with the OnCoat outer layer. Exemplary enveloped viruses or lipid containing viruses contemplated for use herein include: Poxvirus (e.g., vaccinia virus, and the like), Herpes Virus (e.g., herpes simplex, and the like), Rhabdovirus (e.g., Vesicular stomatitis virus, and the like), Coronavirus (e.g., SARS-CoV-2, and the like), Hepadnaviruses, Asfarviridae, Flavivirus, Alphavirus, Togavirus, Hepatitis D, Orthomyxovirus, Paramyxovirus, Bunyavirus, Filovirus, Retroviruses, Alphavirus (alphaviruses), Rubivurus (rubella virus), Flavivirus (Flaviviruses), Pestivirus (mucosal disease viruses), hepatitis C virus, Coronavirus, (Coronaviruses), severe acute respiratory syndrome (SARS), Torovirus, (toroviruses), Arteivirus, (arteriviruses), Paramyxovirus, (Paramyxoviruses), Rubulavirus (rubulavriuses), Morbillivirus (morbillivuruses), Pneumovirinae (the pneumoviruses), Pneumovirus (pneumoviruses), Vesiculovirus (vesiculoviruses), Lyssavirus (lyssaviruses), Ephemerovirus (ephemeroviruses), Cytorhabdovirus (plant rhabdovirus group A), Nucleorhabdovirus (plant rhabdovirus group B), Filovirus (filoviruses), Influenzavirus A, B (influenza A and B viruses), influenza virus C (influenza C virus), (unnamed, Thogoto-like viruses), Bunyavirus (bunyaviruses), Phlebovirus (phleboviruses), Nairovirus (nairoviruses), Hantavirus (hantaviruses), Tospovirus (tospoviruses), Arenavirus (arenaviruses), unnamed mammalian type B retroviruses, unnamed, mammalian and reptilian type C retroviruses, unnamed, type D retroviruses, Lentivirus (lentiviruses), Spumavirus (spumaviruses), Orthohepadnavirus (hepadnaviruses of mammals), Avihepadnavirus (hepadnaviruses of birds), Simplexvirus (simplexviruses), Varicellovirus (varicelloviruses), Betaherpesvirinae (the cytomegaloviruses), Cytomegalovirus (cytomegaloviruses), Muromegalovirus (murine cytomegaloviruses), Roseolovirus (human herpes virus 6, 7, 8), Gammaherpesvirinae (the lymphocyte-associated herpes viruses), Lymphocryptovirus (Epstein-Barr-like viruses), Rhadinovirus (saimiri-ateles-like herpes viruses), Orthopoxvirus (orthopoxviruses), Parapoxvirus (parapoxviruses), Avipoxvirus (fowlpox viruses), Capripoxvirus (sheeppox-like viruses), Leporipoxvirus (myxomaviruses), Suipoxvirus (swine-pox viruses), Molluscipoxvirus (molluscum contagiosum viruses), Yatapoxvirus (yabapox and tanapox viruses), Unnamed, African swine fever-like viruses, Iridovirus (small iridescent insect viruses), Ranavirus (front iridoviruses), Lymphocystivirus (lymphocystis viruses of fish), Togaviridae, Flaviviridae, Coronaviridae, Enabdoviridae, Filoviridae, Paramyxoviridae, Orthomyxoviridae, Bunyaviridae, Arenaviridae, Retroviridae, Hepadnaviridae, Herpesviridae, Poxviridae, and any other lipid-containing, enveloped virus.

In other embodiments, the virus can be selected from the following human and animal pathogens: Ross River virus, fever virus, dengue viruses, Murray Valley encephalitis virus, tick-borne encephalitis viruses (including European and far eastern tick-borne encephalitis viruses, California encephalitis virus, St. Louis encephalitis virus, sand fly fever virus, human coronaviruses 229-E and OC43 and others causing the common cold, upper respiratory tract infection, probably pneumonia and possibly gastroenteritis), human parainfluenza viruses 1 and 3, mumps virus, human parainfluenza viruses 2, 4a and 4b, measles virus, human respiratory syncytial virus, rabies virus, Marburg virus, Ebola virus, influenza A viruses and influenza B viruses, Arenavirus: lymphocytic choriomeningitis (LCM) virus; Lassa virus, human immunodeficiency viruses 1 and 2, or any other immunodeficiency virus, hepatitis B virus, hepatitis C virus, hepatitis G virus, Subfamily: human herpes viruses 1 and 2, herpes virus B, Epstein-Barr virus), (smallpox) virus, cowpox virus, monkeypox virus, molluscum contagiosum virus, yellow fever virus, poliovirus, Norwalk virus, orf virus, and any other lipid-containing, enveloped virus. See, e.g., Mathews, J. gen. Virol. (1975), 27:135-149; which is incorporated herein by reference in its entirety for all purposes.

Also provided herein is a surface-re-engineered virus comprising:
a previously-enveloped-capsid from a naturally occurring enveloped-virus; and an artificial coating layer surrounding the previously-enveloped-capsid.

As used herein, “re-engineered virus” refers to an artificial transducing agent that has a capsid from a previously enveloped virus, wherein the previously-enveloped-capsid is completely coated or completely encapsulated by an artificial coating layer (i.e., artificial coating).

As set forth herein, the artificial coating is selected from the group consisting of: silica, titanium oxide and calcium phosphate. In certain embodiments, the artificial coating is applied by conducting a charge-mediated sol-gel condensation reaction directly onto the surface of the previously-enveloped-capsid. In other embodiments, the artificial coating comprises a silica gel matrix or titanium oxide gel matrix encapsulating the previously-enveloped-capsid. The previously-enveloped-capsid can be either replication-competent or replication-defective.

Also provided herein is a method of re-engineering a virus having a native-envelope, said method comprising:
removing the native-envelope surrounding a capsid from the virus; isolating the previously-enveloped-capsid; and
applying an artificial coating to the previously-enveloped-capsid to form an re-engineered virus.

The various embodiments described above can be combined to provide further embodiments. All of the U.S. patents, U.S. patent application publications, U.S. patent application, foreign patents, foreign patent application and non-patent publications referred to in this specification and/or listed in the Application Data Sheet are incorporated herein by reference, in their entirety to the same extent as if each individual publication, patent, or patent application was specifically and individually indicated to be incorporated by reference.

In other embodiments, the recombinant virus genome or nucleic acid can encode molecular components used for genome editing, for example genome editing nucleases. Such genome editing nucleases can be Cas and related nucleases (e.g., Cas3, Cas9, CasX) for CRISPR, TALE nucleases, zinc finger nucleases, PPR nucleases, or other nucleases. Surface-engineered delivery systems of such embodiments can thus be used in methods of in vivo, in vitro, or ex vivo genome editing.

Aspects of the embodiments can be modified, if necessary to employ concepts of the various patents, application and publications to provide yet further embodiments. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

### Application of ONCoat Technology on Ad26.COV2-S

### Experimental Overview

The objective of this proposed experiment study is to specifically evaluate the application of ONCoat technology with Ad26.COV2-S. This experiment demonstrates the applicability of ONCoat surface engineering technology, specifically with Ad26.COV2-S, and focuses on a number of benefits that ONCoat technology. The experiment is conducted in 3 parts. Application of ONCoat to Ad26.COV2-S is contemplated herein to: 1) prevent the neutralization of Ad26 vector thereby removing any impact of pre-existing immunity to the virus—expanding the population effectively immunized; 2) improve the vaccine efficiency by potentially reducing the vector dose required to elicit protective immunity; 3) reduce potential toxicity that may be related to vector dose and/or immune response to the vector; 4) enable effective boosting since a vector with ONCoat will not be impacted by any pre-existing or primary dose host immunogenicity to the vector particle; and 5) improve vector stability and reducing the cold chain requirements.

### Parts 1 & 2:

The physical characterization, biological activity in the presence and absence of neutralizing antibodies.
1. Initial QC and assay development for Ad26.COV2
2. Surface Engineering of Ad26.COV2 with ONCoat: ONCoat-Ad26-COV2
   a. Optimization of chemistry: Upon coating, the surface charge of the Ad26 is neutralized and the viral suspension is further stabilized as the coating provides steric stabilization. The ONCoat surface is functionalized with folic acid molecules to enable cell uptake and polyethylene glycol polymers to provide steric stability. Folic acid is an essential nutrient; and most human cells uptake folic acid efficiently.
   b. QC
      i. Viral particle quantification is conducted using qPCR
      ii. Optimization of protection assay
      iii. Viral activity evaluation—Transduction & Expression: in vitro—Protein Expression
      iv. Physical characterization: Size (DLS), surface charge (DLS), shape (SEM)
   c. Comparative Studies for Biological Activity: After optimization of the encapsulation procedure, biochemical characterization is performed and cell culture assays comparing material from the original virus stock with the encapsulated virus formulation to evaluate the improvement the surface engineered vector.

The cell culture experiments are used to compare the transduction and expression of SARS-CoV-2 S protein of the reengineered virus with control virus. For this set of experiments, the number of viral particles (determined by qPCR) is matched for both control group (Ad26.COV2) and reengineered group (ONCoat-Ad26-COV2). Experiments are performed both in the presence and absence of neutralizing antibodies against Ad26 vector using various antibody concentrations, of commercially available anti-Ad26 antibodies.

### Part 3:

1. Comparative In vivo administration
   a. Tox & PK & biodistribution are conducted
   b. Tissue antigen expression
   c. Immunological studies
   d. Evaluation of Booster shots/Repeated Administration
      i. Ad26.COV2-S, Ad26.COV2-S booster
      ii. Ad26.COV2-S, DevaCell booster
      iii. DevaCell, DevaCell booster

Ad26.COV2-S at a concentration of ˜1×10¹² vp/ml in cryoprotective buffer at −80° C. is provided in several aliquots (i.e. 0.1 ml) to avoid extra freeze/thaw cycles.

In yet further embodiment, the surface-engineered recombinant virus is a vaccine and is selected from AZD1222 (AstraZeneca), ChAdOx1-nCov19 (Oxford), Ad5-nCoV (CanSino), VSV, Ad26 (J&J), and the like.

What is claimed is:

1. A surface-engineered recombinant virus, said virus comprising:
a recombinant virus having a recombinant genome; and
an artificial coating layer surrounding the recombinant virus,

2. The surface-engineered recombinant virus of claim 1, wherein the artificial coating is selected from the group consisting of: silica, titanium oxide and calcium phosphate.

3. The surface-engineered recombinant virus of claim 1, wherein the virus is selected from the group consisting of: enadenotucirev oncolytic virus, Poxvirus, vaccinia virus, Herpes Virus, herpes simplex virus-1, herpes simplex virus-2, Rhabdovirus, Vesicular stomatitis virus, Coronavirus, SARS-CoV-2, Hepadnaviruses, Asfarviridae, Flavivirus, Alphavirus, Togavirus, Hepatitis D virus, Orthomyxovirus, Paramyxovirus, Bunyavirus, Filovirus, Retrovirus, Noroviruses, adenoviruses, rotaviruses, poliovirus, Picornaviruses, enteroviruses, rhinoviruses, Coxsackie viruses, echoviruses, and hepatitis A virus.

4. The surface-engineered recombinant virus of claim 1, wherein the recombinant virus is oncolytic and is selected from the group consisting of: NG-641 (PsiOxus), and Imlygic (talimogene laherparepvec).

5. The surface-engineered recombinant virus of claim 1, wherein the virus is a vaccine and is selected from AZD1222 (AstraZeneca), ChAdOx1-nCov19 (Oxford), Ad5-nCoV (CanSino), VSV, and Ad26 (J&J).

6. The surface-engineered recombinant virus of claim 1, wherein the virus is replication deficient Ad5 (Human) Adenovirus vector, and wherein the recombinant genome encodes SARS-CoV-2 spike protein and E1 & E3 genes are deleted.

7. The surface-engineered recombinant virus of claim 1, wherein the virus is oncolytic and the virus is VSV.

8. The surface-engineered recombinant virus of claim 1, wherein the artificial coating is applied by conducting a charge-mediated sol-gel condensation reaction directly onto the surface of the recombinant virus.

9. The surface-engineered virus of claim 7, wherein the artificial coating comprises a silica gel matrix or titanium oxide gel matrix encapsulating the recombinant virus.

10. The surface-engineered virus of claim 1, wherein the artificial coating comprises a targeting-ligand selected from the group consisting of: proteins, polysaccharides, aptamers, peptides, oligonucleotides and small molecules.

11. The surface-engineered virus of claim 1, wherein the artificial coating comprises a targeting-ligand selected from the group consisting of: Antibodies, transferrin, Hyaluronic acid, RGD, IL4RPep-1, AS-1411, GBI-10, Folate, anisamide, and phenylboronic acid.

12. The surface-engineered virus of claim 1, wherein the recombinant virus is replication-competent or replication-defective.

13. The surface-engineered virus of claim 1, wherein the recombinant virus has had it native-envelope removed prior to coating with the artificial coating layer.

14. A surface-engineered recombinant virus vaccine, said virus comprising:
a recombinant Ad5 virus having a recombinant genome encoding SARS-CoV-2 spike protein, wherein at least a functional portion of E1 and E3 genes are deleted, or wherein the virus does not contain any native Ad5 viral genes; and
an artificial coating layer encapsulating the recombinant Ad5 virus, wherein said coating layer comprises an effective amount of folate to bind a folate receptor on a cell, and wherein the artificial coating is selected from the group consisting of: silica, titanium oxide and calcium phosphate

15. A method of making a surface-engineered recombinant virus, said method comprising:
producing a recombinant virus having a recombinant genome; and
applying an artificial coating to the recombinant virus, wherein the artificial coating is selected from the group consisting of: silica, titanium oxide and calcium phosphate.

16. The method of claim 14, wherein the artificial coating is applied by conducting a charge-mediated sol-gel condensation reaction directly onto the surface of the recombinant virus.

17. The method of claim 15, wherein the artificial coating comprises a silica gel matrix or titanium oxide gel matrix encapsulating the recombinant virus.

18. The surface-engineered virus of claim 14, wherein the recombinant virus has had its native-envelope removed prior to coating with the artificial coating layer.

19. A method of re-engineering the surface of a virus having a native-envelope, said method comprising:
removing the native-envelope from the virus to isolate a previously-enveloped-capsid;
applying an artificial coating to the previously-enveloped-capsid.

20. The method of claim 18, wherein the envelope virus is selected from the group consisting of: Poxvirus, vaccinia virus, Herpes Virus, herpes simplex virus-1, herpes simplex virus-2, Rhabdovirus, Vesicular stomatitis virus, Coronavirus, SARS-CoV-2, Hepadnaviruses, Asfarviridae, Flavivirus, Alphavirus, Togavirus, Hepatitis D virus, Orthomyxovirus, Paramyxovirus, Bunyavirus, Filovirus, and Retrovirus.

21. The method of claim 18, wherein the native-envelope is removed or delipidated using a detergent and/or an extraction solvent.

22. The method of claim 20, wherein the detergent and/or an extraction solvent is selected from the group consisting of: Glutaraldehyde, chloroform, B-propiolactone, TWEEN-80, and dialkyl or trialkyl phosphates, alcohols, hydrocarbons, amines, ethers, n-butanol, di-isopropyl ether (DIPE), diethyl ether, either alone or in combination.

23. The method of claim 18, wherein the artificial coating is selected from the group consisting of: silica, titanium oxide and calcium phosphate.

24. The method of claim 13, wherein applying the artificial coating further comprises conducting a charge-mediated sol-gel condensation reaction directly onto the surface of the previously-enveloped-capsid.

25. The method of claim 22, wherein the artificial coating comprises a silica gel matrix or titanium oxide gel matrix encapsulating the previously-enveloped-capsid.

26. The method of claim 18, wherein the previously-enveloped-capsid is replication-competent or replication-defective.

27. A surface-re-engineered virus comprising:
a previously-enveloped-capsid from a naturally occurring enveloped-virus; and
an artificial coating layer surrounding the previously-enveloped-capsid.

28. The surface-re-engineered virus of claim 26, wherein the envelope virus is selected from the group consisting of: Poxvirus, vaccinia virus, Herpes Virus, herpes simplex virus-1, herpes simplex virus-2, Rhabdovirus, Vesicular stomatitis virus (VSV), Coronavirus, SARS-CoV-2, Hepadnaviruses, Asfarviridae, Flavivirus, Alphavirus, Togavirus, Hepatitis D virus, Orthomyxovirus, Paramyxovirus, Bunyavirus, Filovirus, and Retrovirus.

29. The surface-re-engineered virus of claim 26, wherein the artificial coating is selected from the group consisting of: silica, titanium oxide and calcium phosphate.

30. The surface-re-engineered virus of claim 26, wherein the artificial coating is applied by conducting a charge-mediated sol-gel condensation reaction directly onto the surface of the previously-enveloped-capsid,

31. The surface-re-engineered virus of claim 29, wherein the artificial coating comprises a silica gel matrix or titanium oxide gel matrix encapsulating the previously-enveloped-capsid.

32. The surface-re-engineered virus of claim 26, wherein the previously-enveloped-capsid is replication-competent or replication-defective.

33. A method of re-engineering a virus having a native-envelope, said method comprising:
removing the native-envelope surrounding a capsid from the virus;
isolating the previously-enveloped-capsid; and
applying an artificial coating to the previously-enveloped-capsid.

34. A composition comprising;
a capsid from a native envelope-virus, wherein the capsid is devoid of its native envelope; and
an artificial coating-layer, wherein the coating-layer encapsulates the capsid.

35. The composition of claim 33, wherein the envelope-virus is selected from the group consisting of: Herpesviruses, Poxviruses (e.g., vaccinia virus), Hepadnaviruses, Asfarviridae, Flavivirus, Alphavirus, Togavirus, Coronavirus, Hepatitis D, Orthomyxovirus, Paramyxovirus, Rhabdovirus, Bunyavirus, Filovirus, and Retroviruses.

36. The composition of claim 33, wherein the coating-layer protects the capsid from immune recognition and neutralization during therapy.

37. The composition of claim 33, wherein the coating-layer further comprises binding agents on its surface that changes the infectivity and/or biological activity of the native envelope-virus.

38. A method of making the composition of claim 33, comprising removing the envelope of an envelope virus to produce an envelope-free-capsid; and encapsulating the envelope-free-capsid with an artificial coating-layer.

39. A surface-engineered delivery system, said system comprising:
a payload; and
an artificial coating layer surrounding the payload.

40. The surface-engineered delivery system, wherein the payload is selected from a recombinant virus or a nucleic acid.

41. The surface-engineered delivery system, wherein the artificial coating is selected from the group consisting of: silica, titanium oxide and calcium phosphate
